(19) United States
(12) Patent Application Publication
(10) Pub. No.: US 2014/0227793 A1
(43) Pub. Date: Aug. 14, 2014

(54) COMPOSITIONS AND METHODS FOR GLYCAN SEQUENCING

(71) Applicant: CALIFORNIA INSTITUTE OF TECHNOLOGY, Pasadena, CA (US)

(72) Inventors: Jinshan Gao, Pasadena, CA (US); Jesse L. Beauchamp, La Canada Flintridge, CA (US)

(21) Appl. No.: 14/181,622

(22) Filed: Feb. 14, 2014

Related U.S. Application Data
(60) Provisional application No. 61/764,975, filed on Feb. 14, 2013.

(51) Int. Cl. G01N 33/50; C07D 401/12

(52) U.S. Cl. CPC G01N 33/50; C07D 401/12
USPC 436/94; 546/193; 536/124

(57) ABSTRACT

Compositions and methods directed to a glycan reagent for depolymerization of a glycan having a reducing end, the glycan reagent being represented by the formula MCX, in which M is a glycan coupling group selected from oxylamines and hydrazides; C is a fixed charge group or a basic group having a proton affinity of at least 210 kcal/mol; and X is hydrogen or a free radical initiator chemically coupled to C.

### CROSS-REFERENCE TO RELATED APPLICATION(S)

The present application claims priority to and the benefit of U.S. Provisional Application Ser. No. 61/764,975 filed on Feb. 14, 2013, the entire contents of which are incorporated herein by reference.

### STATEMENT REGARDING FEDERALLY SPONSORED RESEARCH OR DEVELOPMENT

This invention was made with government support under CHE0416381 awarded by the National Science Foundation. The government has certain rights in the invention.

### FIELD

This disclosure is directed to glycan reagents and the use of the glycan reagents to determine the structure of target glycans having a reducing terminus.

### BACKGROUND

Glycans are one of the four families of structurally related macromolecules that comprise living organisms, along with nucleic acids, proteins, and lipids. However, unlike DNA, RNA, and proteins, which possess predominantly linear structures comprising a limited number of subunits with defined stereochemistry, glycans may exhibit complicated branched structures with a large number of subunits having both structural and stereochemical diversity. As a result, the field of glycomics is much less developed than genomics and proteomics. Nevertheless, genetic and biochemical studies over the past several decades have established the importance of glycans in many fields, including various aspects of health, such as immunity response, inflammation signaling, and disease prevention, as well as green energy production and materials fabrication. Therefore, understanding the structure and function of glycans will complement and strengthen other areas of research.

Mass spectrometry (MS), noted for its minimal sample consumption, high sensitivity, and short acquisition time, has been widely employed for structural characterization of glycans. Moreover, mass spectrometers allowing for tandem mass spectrometry (MSⁿ) have been used extensively as indispensable tools for glycan structural analysis. Ionization efficiency is especially important for the MSⁿ experiments to achieve good sensitivity and wide dynamic ranges. However, due to lack of strongly basic sites for protonation, glycans are traditionally difficult to ionize. (Edge et al. Nature 1992, 358, 693-694, the entire contents of which are herein incorporated by reference.)

Using suitably ionized glycans, many techniques exist for effecting dissociation processes that provide structural information. Low-energy collision-induced dissociation (CID) typically generates glycosidic bond cleavage when applied to glycans. Infrared multiphoton dissociation (IRMPD), another slow-heating fragmentation method, gives results similar to low-energy CID. (Harvey, D. J. J. Am. Soc. Mass Spectrom. 2001, 12, 926-937; Adamson, J. T.; Hakansson, K. Anal. Chem. 2007, 79, 2901-2910; and Xie, Y. M.; Lebrilla, C. B. Anal. Chem. 2003, 75, 1590-1598, the entire contents of all of which are herein incorporated by reference.) High-energy CID and vacuum ultraviolet multiphoton dissociation are unavailable on many modern instruments, even though they can generate more cross-ring cleavages than low-energy CID and IRMPD. More recently, techniques including electron capture dissociation (ECD), electron detachment dissociation (EDD), and electron transfer dissociation (ETD) have been demonstrated to provide extensive and complementary information about glycan structure. (Zhang et al., J. Proteome Res. 2009, 8, 734-742; Budnik et al., Anal. Chem. 2003, 75, 5994-6001. Zhao et al., J. Am. Soc. Mass Spectrom. 2008, 19, 138-150; Wolff et al., Anal. Chem. 2010, 82, 3460-3466; and Han et al., C. J. Am. Soc. Mass Spectrom. 2011, 22, 997-1013, the entire contents of all of which are herein incorporated by reference.)

In contrast to the often unpredictable dissociation pathways and yields associated with the above techniques, natural enzymes excel in depolymerizing glycans into their components, often in a systematic and predictable manner, by taking advantage of acid-base catalysis to achieve selective cleavage of the glycosidic bond. In fact, enzymatic structural analysis of glycans employing a set of highly specific exoglycosidases, sequentially or in a matrix array, has proven to be a powerful analytical tool for the determination of sequence, linkage type, and anomeric configuration. However, this method requires highly pure samples, fully completed hydrolysis, and lengthy enzymatic incubation periods. In addition, certain glycosidic bonds, particularly those between two glucose residues, are highly resistant to enzymatic degradation due to their high stability. (Edge et al., supra; and Wolfenden et al., J. Am. Chem. Soc. 2008, 130, 7548-7549, the entire contents of both of which are herein incorporated by reference.)

The efficiency of these natural enzymes provides an impetus to the development of biomimetic reagents that, when combined with MS, attempt in part to replicate their chemistry while eliminating the shortcomings of a purely enzymatic approach to glycan sequencing.

### SUMMARY

In embodiments of the present invention, a glycan reagent for depolymerization of a glycan having a reducing end is represented the formula MCX, wherein M is a glycan coupling group selected from oxylamines and hydrazides; C is a fixed charge group or a basic group having a proton affinity of 210 kcal/mol; and X is hydrogen or a free radical initiator.

In some embodiments, the glycan reagent is represented by one of the following formulas:

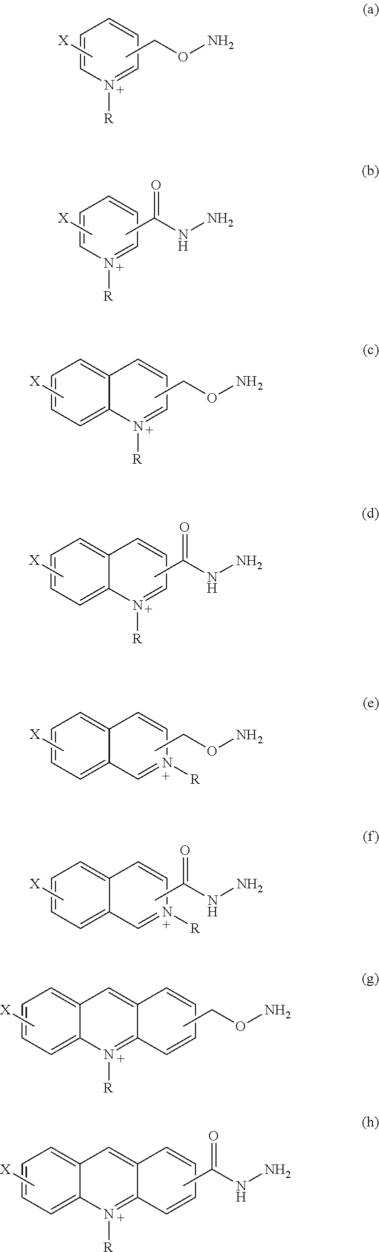

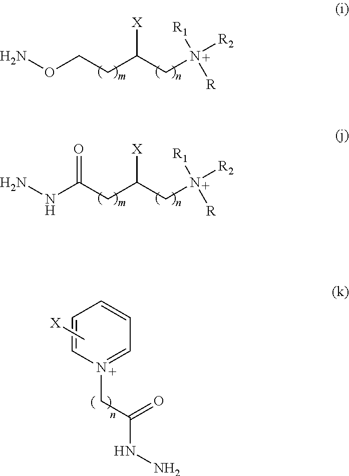

In the above formulas, X is hydrogen or a free radical initiator, R is hydrogen, an alkyl, or an alkoxy, R₁ and R₂ are each independently an alkyl, and m and n are each independently an integer.

In some embodiments, a method of depolymerizing a glycan having a reducing terminus includes conjugating the glycan reagent of claim 1 to the reducing terminus of the glycan to form a derivatized glycan, ionizing the derivatized glycan to form an ionized derivatized glycan, and dissociating the ionized derivatized glycan to form fragment glycan ions.

In some embodiments, a method of depolymerizing a glycan having a reducing terminus includes conjugating a glycan reagent to the reducing terminus of the glycan to form a derivatized glycan, isolating the derivatized glycan to form an isolated derivatized glycan, ionizing the isolated derivatized glycan to form an ionized derivatized glycan, and dissociating the ionized derivatized glycan to form fragment glycan ions.

In some embodiments, a method of identifying subunit connectivity of a glycan having a reducing terminus includes conjugating a glycan reagent represented by one of structures (a) through (j) to the reducing terminus of the glycan to form a derivatized glycan, ionizing the derivatized glycan to form an ionized derivatized glycan, dissociating the ionized derivatized glycan to form fragment glycan ions, and analyzing the fragment glycan ions to determine the subunit connectivity of the glycan:

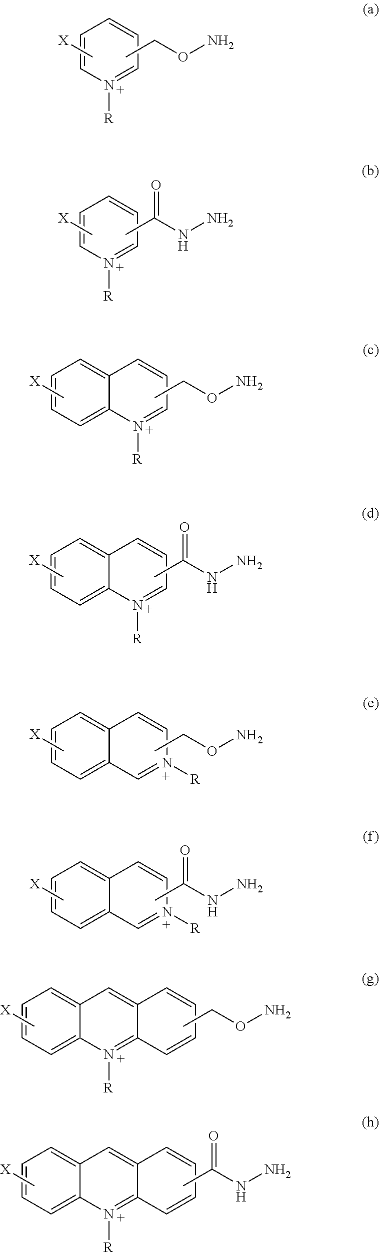

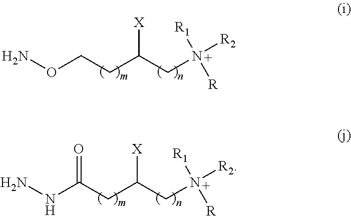

In the above structures, X is hydrogen, R is hydrogen, R₁ and R₂ are each independently an alkyl.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic showing (on the left) an example of the acid-base chemistry of the proton reagent for acid-catalyzed glycan sequencing (PRAGS), and (on the right) an example of the radical chemistry of a free radical activated glycan sequencing (FRAGS) reagent, according to embodiments of the present invention.

FIG. 2 is a schematic showing the derivation of glycan fragment ions and nomenclature according to the Domon and Costello nomenclature as further described in this disclosure.

FIG. 3 is an MS¹ spectrum of FRAGS-derivatized maltoheptaose, according to embodiments of the present invention.

FIG. 4A shows the observed fragmentation patterns following CID of singly protonated PRAGS-derivatized maltoheptaose, according to embodiments of the present invention.

FIG. 4B is a CID spectrum corresponding to the singly protonated PRAGS-derivatized maltohelptaose as depicted in FIG. 4A, in which the labeled Parent Ion refers to the protonated molecular ion, according to embodiments of the present invention.

FIG. 4C shows the observed fragmentation patterns following CID of FRAGSII-derivatized maltoheptaose, according to embodiments of the present invention.

FIG. 4D is a CID spectrum corresponding to the singly protonated FRAGS II-derivatized maltohelptaose as depicted in FIG. 4C, in which the labeled Parent Ion refers to the protonated molecular ion, according to embodiments of the present invention.

FIG. 4E shows the observed fragmentation patterns following CID of FRAGS I-derivatized maltoheptaose, according to embodiments of the present invention.

FIG. 4F is a CID spectrum corresponding to the singly protonated FRAGS I-derivatized maltohelptaose as depicted in FIG. 4E, in which the labeled Parent Ion refers to the protonated molecular ion, according to embodiments of the present invention.

FIG. 5A is a CID spectrum for a PRAGS-derivatized maltoheptaose, in which the labeled Parent Ion refers to the protonated molecular ion, according to embodiments of the present invention.

FIG. 5B is a CID spectrum for a Girard's T (GT)-derivatized maltoheptaose, in which the labeled Parent Ion refers to the protonated molecular ion, according to embodiments of the present invention.

FIG. 6A shows the observed fragmentation patterns following CID of [M+H-TEMPO]+ ions of maltose, according to embodiments of the present invention.

FIG. 6B is a CID spectrum corresponding to the [M+H-TEMPO]+ ions of maltose as depicted in FIG. 6A, according to embodiments of the present invention.

FIG. 6C is a CID spectrum of a PRAGS-derivatized maltose, according to embodiments of the present invention.

FIG. 6D shows the observed fragmentation patterns following CID of [M+H-TEMPO]+ ions of cellobiose, according to embodiments of the present invention.

FIG. 6E is a CID spectrum corresponding to the [M+H-TEMPO]+ ions of cellobiose as depicted in FIG. 6D, according to embodiments of the present invention.

FIG. 6F is a CID spectrum of a PRAGS-derivatized cellobiose, according to embodiments of the present invention.

FIG. 6G shows the observed fragmentation patterns following CID of [M+H-TEMPO]+ ions of lactose, according to embodiments of the present invention.

FIG. 6H is a CID spectrum corresponding to the [M+H-TEMPO]+ ions of lactose as depicted in FIG. 6G, according to embodiments of the present invention.

FIG. 6I is a CID spectrum of a PRAGS-derivatized lactose, according to embodiments of the present invention.

FIG. 6J shows the observed fragmentation patterns following CID of [M+H-TEMPO]+ ions of nigerose, according to embodiments of the present invention.

FIG. 6K is a CID spectrum corresponding to the [M+H-TEMPO]+ ions of nigerose as depicted in FIG. 6J, according to embodiments of the present invention.

FIG. 6L is a CID spectrum of a PRAGS-derivatized nigerose, according to embodiments of the present invention.

FIG. 7A shows a CID spectrum of FRAGS II-derivatized lactose which upon collisional activation generate the [M+H-TEMPO]+ ions of lactose of FIG. 6G, according to embodiments of the present invention.

FIG. 7B shows a CID spectrum of FRAGS II-derivatized maltose, which upon collisional activation generate the [M+H-TEMPO]+ ions of maltose of FIG. 6A, according to embodiments of the present invention.

FIG. 8 is a graph showing branching ratios (Fraction of total fragment yield) of different fragmentation patterns of maltose, cellobiose, lactose, and nigerose as shown from left to right, respectively, according to embodiments of the present invention.

FIG. 9A shows the fragmentation patterns observed following CID of singly protonated PRAGS-derivatized LNDFH II (Lacto-N-difucohexaose II), according to embodiments of the present invention.

FIG. 9B is a CID spectrum corresponding to the PRAGS-derivatized LNDFH as depicted in FIG. 8A, according to embodiments of the present invention.

FIG. 10 is a diagram of glycan deconstruction (DECON diagram) for LNDFH II in which the precursor ion (m/z 1106) is subjected to MS² to generate a series of ions with m/z values from 287 to 960 and the MS² product ions are isolated and subjected to CID to generate MS³ product ions, according to embodiments of the present invention.

FIG. 11A shows a first part of the fragmentation patterns observed following CID of singly protonated FRAGS II-derivatized LNDFH II, according to embodiments of the present invention.

FIG. 11B shows a second part of the fragmentation patterns observed following CID of singly protonated FRAGS II-derivatized LNDFH II, according to embodiments of the present invention.

FIG. 11C is a CID spectrum corresponding to the singly protonated FRAGS II-derivatized LNDFH II depicted in FIGS. 10A and 10B, in which peaks marked with asterisks are the product ions corresponding to the glycosidic bond cleavages from the parent ion, according to embodiments of the present invention.

FIG. 11D is a CID spectrum corresponding to the FRAGSI-derivatized LNDFH II, according to embodiments of the present invention.

FIG. 11E is a schematic showing the structures of LNDFH I and LNDFH II.

FIG. 11F are the CID spectra of FRAGS I-derivatized LNDFH I and FRAGS I-derivatized LNDFH II, according to embodiments of the present invention.

FIG. 12A shows the fragmentation patterns observed following CID of singly-protonated PRAGS-derivatized Lewis-Y tetrasaccharide, according to embodiments of the present invention.

FIG. 12B is a CID spectrum corresponding to the singly-protonated PRAGS-derivatized Lewis-Y tetrasaccharide depicted in FIG. 12A, according to embodiments of the present invention.

FIG. 12C shows the fragmentation patterns observed following CID of singly-protonated FRAGS II-derivatized Lewis-Y tetrasaccharide, according to embodiments of the present invention.

FIG. 12D is a CID spectrum corresponding to the singly-protonated FRAGS II-derivatized Lewis-Y tetrasaccharide depicted in FIG. 12C, according to embodiments of the present invention.

FIG. 13A shows the fragmentation patterns observed following CID of Z_{3HH}+Z_{3HL}+H ion of LNDFH II, according to embodiments of the present invention.

FIG. 13B is a CID spectrum corresponding to the Z_{3HH}+Z_{3HL}+H ion of LNDFH II as depicted in FIG. 13A, according to embodiments of the present invention.

FIG. 14A shows a CID spectrum corresponding to RS-FRAGS II-derivatized LNFDI, according to embodiments of the present invention.

FIG. 14B shows a CID spectrum corresponding to RS-FRAGS II-derivatized LNFDII, according to embodiments of the present invention.

FIG. 15 is a schematic showing an example protocol for isolating, derivatizing and fragmenting and analyzing N-glycans from a glycoprotein using RS-FRAGS II reagent, according to embodiments of the present invention.

FIGS. 16A, 16B, and 16C show CID spectra of RS-FRAGS II-derivatized N-glycans from RNase B, according to embodiments of the present invention.

FIG. 17 is a graph showing the R—X bond dissociation enthalpies (BDEs) (ΔH₂₉₈) for α-1-O-methyl-D-glucopyranose in which for the M05 and M06 density functionals, the uncertainties of the BDEs are estimated to be 0.5 to 1.0 kcal/mol, as further described in this disclosure.

FIG. 18A is a ¹H NMR spectrum of methyl 5-(bromomethyl)nicotinate, according to embodiments of the present invention.

FIG. 18B is a ¹³C NMR spectrum of methyl 5-(bromomethyl)nicotinate, according to embodiments of the present invention.

FIG. 19A is a ¹H NMR spectrum of methyl 5-(((2,2,6,6-tetramethylpiperidin-1-yl)oxy)methyl)nicotinate, according to embodiments of the present invention.

FIG. 19B is a ¹³C NMR spectrum of methyl 5-(((2,2,6,6-tetramethylpiperidin-1-yl)oxy)methyl)nicotinate, according to embodiments of the present invention.

FIG. 20A is a ¹H NMR spectrum of (5-(((2,2,6,6-Tetramethylpiperidin-1-yl)oxy)methyl)pyridin-3-yl)methanol, according to embodiments of the present invention.

FIG. 20B is a ¹³C NMR spectrum of (5-(((2,2,6,6-Tetramethylpiperidin-1-yl)oxy)methyl)pyridin-3-yl)methanol, according to embodiments of the present invention.

FIG. 21A is a ¹H NMR spectrum of 2-((5-(((2,2,6,6-Tetramethylpiperidin-1-yl)oxy)methyl)pyridin-3-yl)methoxy)isoindoline-1,3-dione, according to embodiments of the present invention.

FIG. 21B is a ¹³C NMR spectrum of 2-((5-(((2,2,6,6-Tetramethylpiperidin-1-yl)oxy)methyl)pyridin-3-yl)methoxy)isoindoline-1,3-dione, according to embodiments of the present invention.

FIG. 22A is a ¹H NMR spectrum of O-((5-(((2,2,6,6-tetramethylpiperidin-1-yl)oxy)methyl)pyridin-3-yl)methyl)hydroxylamine, according to embodiments of the present invention.

FIG. 22B is a ¹³C NMR spectrum of O-((5-(((2,2,6,6-tetramethylpiperidin-1-yl)oxy)methyl)pyridin-3-yl)methyl)hydroxylamine, according to embodiments of the present invention.

FIG. 23A is a ¹H NMR spectrum of O-(pyridin-3-ylmethyl)hydroxylamine, according to embodiments of the present invention.

FIG. 23B is a ¹³C NMR spectrum of O-(pyridin-3-ylmethyl)hydroxylamine, according to embodiments of the present invention.

### DETAILED DESCRIPTION

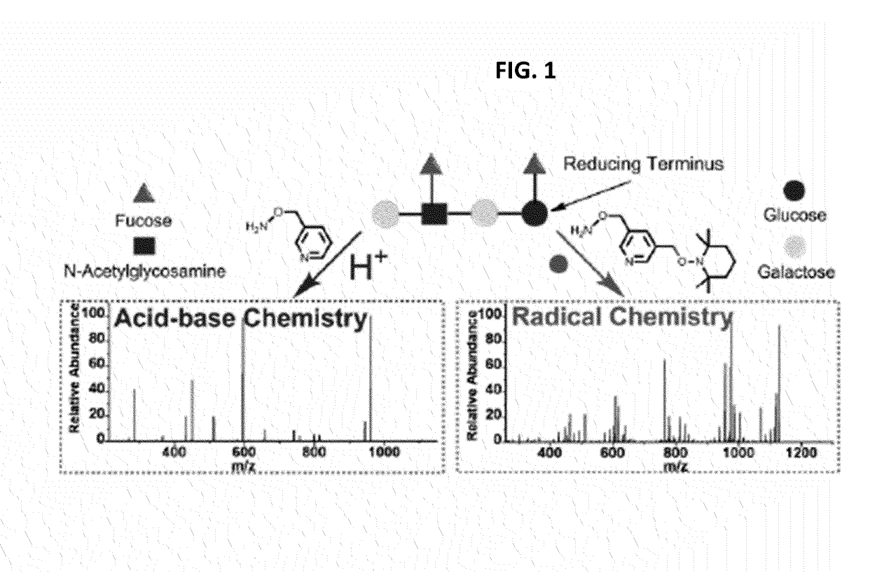

Aspects of the present invention are directed to a glycan reagent that utilizes acid-base chemistry and/or free radical chemistry for depolymerization and sequencing of a glycan having a reducing terminus as depicted in FIG. 1.

As used herein, “depolymerization” and “fragmentation” are used interchangeably and with respect to a glycan, refer to the conversion of the glycan into its subunits. Depolymerization using a glycan reagent as disclosed herein is ordered, and therefore, the depolymerization of the glycan allows for determination of the sequence of the glycan subunits. As such, sequencing of a glycan is the observation of the depolymerization. Additionally, “sequencing of a glycan,” “glycan sequencing,” and “glycan determination” are used interchangeably to refer to the elucidation of a glycan structure resulting from depolymerization (or fragmentation) of the glycan.

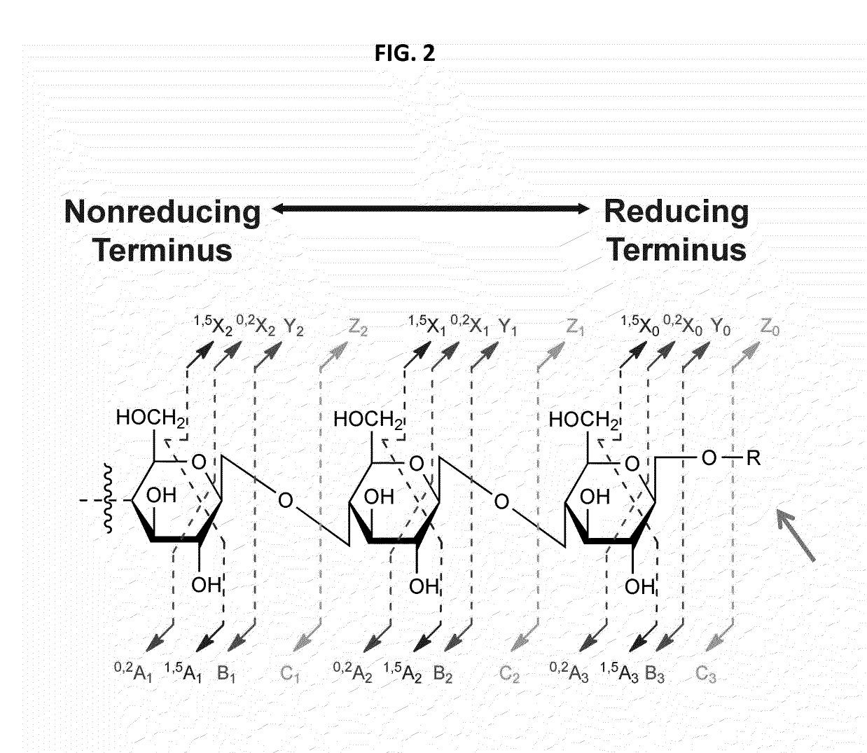

As used herein, all product ions are classified according to the Domon and Costello nomenclature (Domon, B.; Costello, C. E. Glycoconjugate J. 1988, 5, 397-409, the entire contents of which are herein incorporated by reference). The Domon and Costello nomenclature for product ions is shown schematically in FIG. 2, except MH-TEMPO-CH₅O₂ (e.g., H₂O+HOCH₂.) and Y—CH₅O₂, which were not previously reported and are defined herein as n ions. More specifically, ions retaining the charge on the nonreducing terminus are named A (cross-ring), and B and C (glycosidic) while those ions retaining the charge on the reducing terminus are named X (cross-ring), and Y and Z (glycosidic). Letters L and H are employed to differentiate a branched glycan wherein L indicates the lighter branch while H indicates the heavier branch. As shown in the drawings, peaks resulting from different cleavages are labeled in different colors; C1-O glycosidic bond cleavages are labeled in red, O-Cx (x can be 2, 3, 4, 5, 6) glycosidic bond cleavages are labeled in green, 1,5-cross-ring cleavages are labeled in blue, and 0,2-cross-ring cleavages are labeled in purple.

In some embodiments of the present invention, a glycan reagent for ordered depolymerization of a glycan having a reducing terminus includes a compound represented by the formula M-C—X, in which M is a moiety capable of coupling to the reducing end of the glycan, C is a moiety having a fixed charge, and X is either hydrogen (H) or a free radical source (e.g., a free radical initiator). In some embodiments, the glycan coupling group M may be selected from oxylamine groups (—ONH₂) and hydrazides (—NHNH₂). In some embodiments, C is a fixed charged group or a basic group having a proton affinity of at least 210 kcal/mol. When C is a fixed charge group, the charge remains on the reagent when coupled to the reducing end of the glycan. For example, the C moiety includes: cationic species with and without a labile proton; anionic species that have conjugate bases of neutral acids (e.g., proton acceptors) and anionic species that have a fixed negative charge. In some embodiments the C moiety of the MCX reagent has a proton affinity of at least 210 kcal/mol. For example, the free radical source X includes any suitable free radical initiator that is chemically coupled to C. Non-limiting examples of free radical initiators include peroxides, nitrites, alkoxyamines, and azo compounds. Specific examples of free radical initiators include TEMPO and 4,4-azobis(4-cyanopentanoic acid) (Vazo 68, Dupont).

In some embodiments of the present invention, the glycan reagent of the present invention utilizes acid-base depolymerization, free radical activated depolymerization, or a combination of both acid-base and free radical chemistries. An example of an acid-base catalyzed glycan depolymerization compound is referred to as Proton Reagent for Acid-catalyzed Glycan Sequencing (PRAGS). An example of a free radical based glycan depolymerization compound is referred to as Free Radical Activated Glycan Sequencing I (FRAGS I), and an example of an acid-base and free radical glycan depolymerization compound is referred to as Free Radical Activated Glycan Sequencing II (FRAGS II), as shown in FIG. 1.

Similar to enzymatic glycosidic bond cleavage, PRAGS employs acid-base chemistry to effect selective C1-O glycosidic bond cleavage (Y ions), with the charge retained on the reducing terminus. With both a labile proton and radical precursor, collisional activation of FRAGS II-derivatized glycans yields abundant cross-ring as well as glycosidic bond cleavages, resulting from both free radical and acid-base chemistry, with charge retention at the reducing terminus. Additionally FRAGS I-derivatized glycans eliminate the acid-base reaction, using only free-radical chemistry. The difference between FRAGS I and FRAGS II fragmentation is evidenced by the disappearance of the Y* ion and Y+Y ion, thereby decreasing the complexity of the spectra while keeping all the essential fragmentation patterns for the structure determination of glycans. All major fragments are products of Y- and Z-type glycosidic bond cleavages, and ^{1,5}X cross-ring cleavages. Additionally, the specific fragmentation pattern (Z+Z+H), resulting from the free radical chemistry of the FRAGS I, is observed only at the branch site, providing the information to confirm presence of the branch structure.

Example compounds for each of PRAGS, FRAGS I, and FRAGS II reagents are shown below.

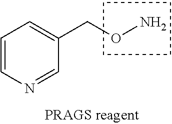

An example of a Proton Reagent for Acid-catalyzed Glycan Sequencing (PRAGS) reagent is shown above, in which the glycan coupling group M is O—NH₂ as shown in the dotted box. A PRAGS reagent does not include a free radical source group, and as such, X is hydrogen. In the above PRAGS example, the fixed charge group C is methylpyridine.

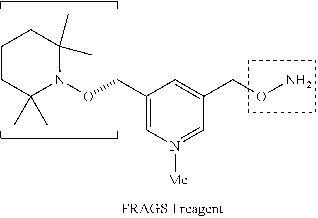

An example of a FRAGS I reagent is shown above in which the glycan coupling group M is O—NH₂ as shown in the dotted box. In this example, the fixed charge group C is a N-methyl-methylpyridinium ion group. The methylation of the pyridine N (nitrogen) atom eliminates the acid-base reaction found in PRAGS and FRAGS II compounds. In the FRAGS I example above, X is (2,2,6,6-Tetramethylpiperidin-1-yl)oxy (TEMPO), shown above in brackets. The bond that is cleaved to produce a free radical between N-methyl-methylpyridinium and TEMPO is shown in bold.

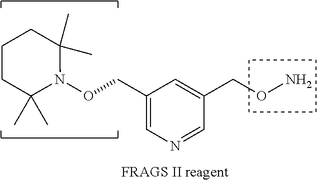

An example of a FRAGS II reagent is shown above in which the glycan coupling group M is O—NH2 as shown in the dotted box. In this example, the fixed charge group C is a methylpyridine and X is TEMPO, shown above in brackets. The bond that is cleaved to produce a free radical between N-methylpyridinium and TEMPO is shown in bold.

In some embodiments of the present invention, the glycan reagent may be a PRAGS, FRAGS I or FRAGS II compound. Non-limiting examples of PRAGS, FRAGS I and FRAGS II compounds are represented by the following structures:

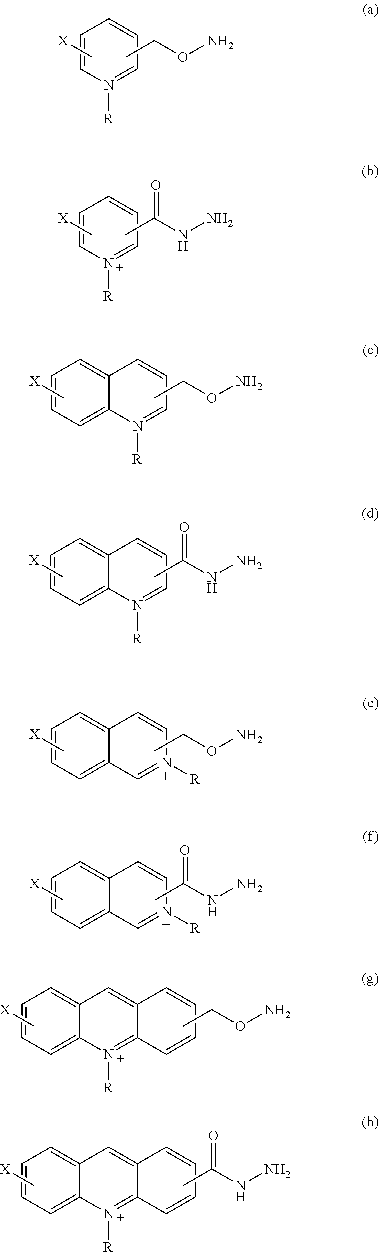

For a PRAGS reagent, for each of structures (a) through (h) above, X is hydrogen and R is hydrogen. For a FRAGS I reagent, for each of structures (a) through (i) above, X is a free radical initiator (e.g., TEMPO-CH₂) and R is an alkyl group or an alkoxy group. For a FRAGS II reagent, for each of structures (a) through (i) above, X is a free radical initiator (e.g., TEMPO-CH₂) and R is hydrogen. For a FRAGS I reagent, for each of structures (a) through (i) above, X is a free radical initiator (e.g., TEMPO-CH₂) and R is alkyl or alkoxy. For PRAGS, FRAGS I, and FRAGS II reagents represented by structure (i), n is any integer so long as the radical group interacts with the glycan and does not react with the reagent itself. In some embodiments n is an integer from 1 to 3.

In some embodiments of the present invention, the glycan reagent may be a PRAGS, FRAGS I or FRAGS II compound represented by one of the following structures:

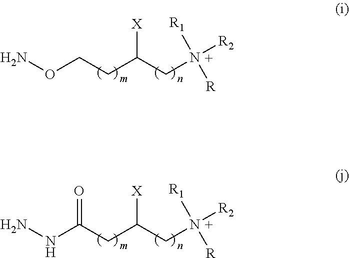

For a PRAGS reagent, for each of structures (i) and (j) above, X is hydrogen, R is hydrogen, and R₁ and R₂ are each independently an alkyl. For a FRAGS I reagent, for each of structures (i) and (j) above, X is a free radical initiator (e.g., TEMPO-CH₂), R is alkyl or alkoxy, and R₁ and R₂ are each independently an alkyl. For a FRAGS II reagent, for each of structures (i) and (j) above, X is a free radical initiator (e.g., TEMPO-CH₂), R hydrogen, and R₁ and R₂ are each independently an alkyl. For PRAGS, FRAGS I, and FRAGS II reagents represented by structures (i) or (j), both m and n are each independently any integer so long as the radical group interacts with the glycan and does not react with the reagent itself. In some embodiments m and n are each independently an integer from 1 to 3.

In some embodiments of the present invention, the glycan reagent may be a FRAGS I reagent represented by the following structure:

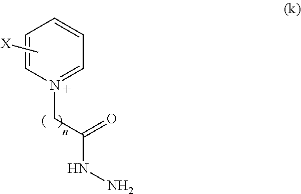

For a FRAGS I reagent, structure (k) and (j) above, X is a free radical initiator (e.g., TEMPO-CH₂) and both m and n are independently any integer so long as the radical group interacts with the glycan and does not react with the reagent itself. In some embodiments m and n are independently are each independently an integer from 1 to 3.

### Synthesis of MCX-Derivatized Glycans.

According to embodiments of the present invention, glycan reagents represented by the structural formula MCX may be synthesized following methods known in the art. Certain specific and exemplary synthetic methods are disclosed herein (Examples 1 and 8), but the present invention is not limited thereto. In some embodiments of the present invention, an MCX glycan reagent (e.g., a PRAGS, FRAGS I, or FRAGS II reagent) is coupled to a glycan having a reducing terminus. Coupling of an MCX reagent to a glycan may be carried out by methods known in the art, certain examples of which are disclosed herein (Example 1), but the present invention is not limited thereto. The coupling of an MCX reagent to a glycan results in an “MCX-derivatized glycan”, (e.g., PRAGS-derivatized glycan, FRAGS I-derivatized glycan, or FRAGS II-derivatized glycan). The MCX-derivatized glycan may also be referred to as an “MCX-glycan conjugate”.

### Ionization and Fragmentation of Derivatized Glycan

In some embodiments of the present invention, the MCX-derivatized glycan is ionized to form an ionized derivatized glycan. The ionized derivatized glycan is subsequently dissociated to form fragmented glycan product ions. For example, the MCX-derivatized glycan may be ionized by electrospray ionization (ESI), followed by collision-induced dissociation (CID), followed by analysis using mass spectrometry. In some embodiments of the present invention, a second CID step is performed. For example, a second CID step may be performed on a product ion generated from the first CID step.

### Glycan Deconstruction (DECON) Diagram

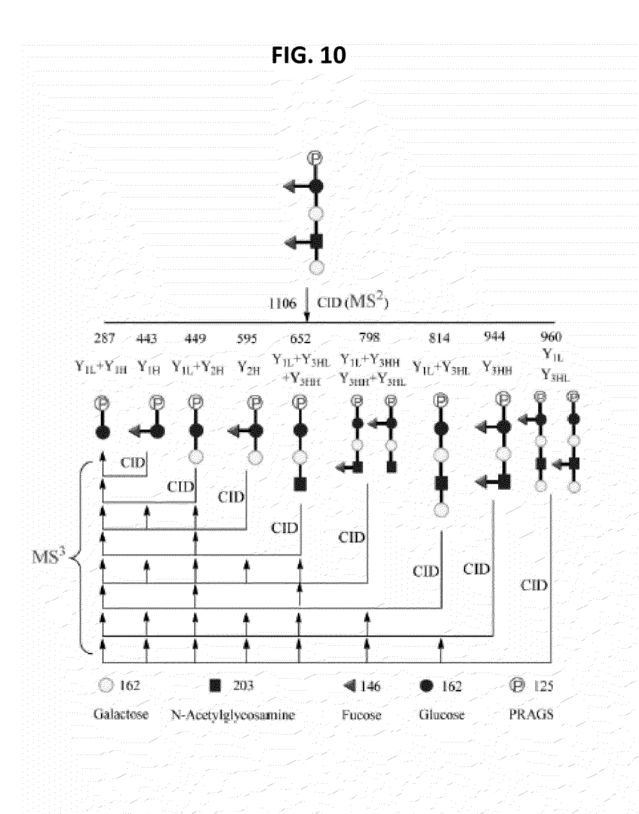

In some embodiments, a method of deconstructing the glycans to identify subunit connectivity includes derivatizing a glycan with a PRAGS reagent followed by fragmentation as disclosed herein. A subunit as disclosed herein refers to a monosaccharide. Because the proton is retained in the PRAGS reagent, subsequent to the first glycosidic bond cleavage, repeat glycosidic bond cleavage reactions are possible. Subsequent bond cleavage reactions allow for a deconstruction of the glycan, thereby producing a DECON diagram. An example of a DECON diagram is shown in FIG. 10. As shown, the PRAGS reagent systematically deconstructs the glycan, revealing subunit connectivity. FIG. 10 visually summarizes the repeated MSⁿ results for LNDFH II.

### Isolation of MCX-Derivatized Glycan

In some embodiments of the present invention, a glycan composition includes an MCX reagent coupled to a support or an affinity tag. Using a support or an affinity tag, derivatized glycans may be effectively isolated for analysis. For example, in order to determine the glycan structure of a selected glycoprotein, the glycans are cleaved from the glycoprotein and then derivatized with an MCX reagent. An affinity tag coupled to the MCX reagent allows for isolation of the derivatized glycans from the protein using the corresponding moiety for the selected support or affinity tag. After isolation of the derivatized glycan and prior to fragmentation, the support or affinity tag is cleaved from the derivatized glycan. In some embodiments of the present invention, any suitable support or affinity tag may be coupled to the MCX reagent. Suitable supports and affinity tags are those which do not inhibit the coupling of the MCX reagent to the glycan and those which can be removed. Non-limiting examples of supports and affinity tags include resins, biotin, and histidine. Coupling of the support or affinity tag is performed using known methods. In some embodiments of the present invention, the MCX reagent is coupled to a resin support, an example using sepharose resin is described herein (Example 5), but the present invention is not limited thereto. Any suitable resin support may be used as would be understood by those having ordinary skill in the art. In some embodiments, the MCX reagent is coupled to an affinity tag such as biotin or histidine, as disclosed in U.S. patent application Ser. No. 13/135,543, the entire contents of which are herein incorporated by reference.

In some embodiments, glycans to be derivatized are first enzymatically removed from a glycoprotein. Enzymatic removal of glycans from glycoproteins, for example, N-glycans, is possible using PNGase F (Peptide-N-Glycosidase F) or Endo H (endonuclease H), as described in Maley et al. 1989, Anal. Biochem. 180: 195-204 PMID: 2510544, the entire contents of which are herein incorporated by reference. For O-linked glycans, a combination of O-glycosidase for core 1 and core 3 glycans in combination with exoglycosidases will allow for removal of large O-glycans as described in Koutsioulis et al., 2008, Glycobiology, 18, 799-805, PMID: 18635885, the entire contents of which are herein incorporated by reference.

The following Examples are presented for illustrative purposes only, and do not limit the scope or content of the present application.

### Example 1

### Synthesis of FRAGS and PRAGS Reagents

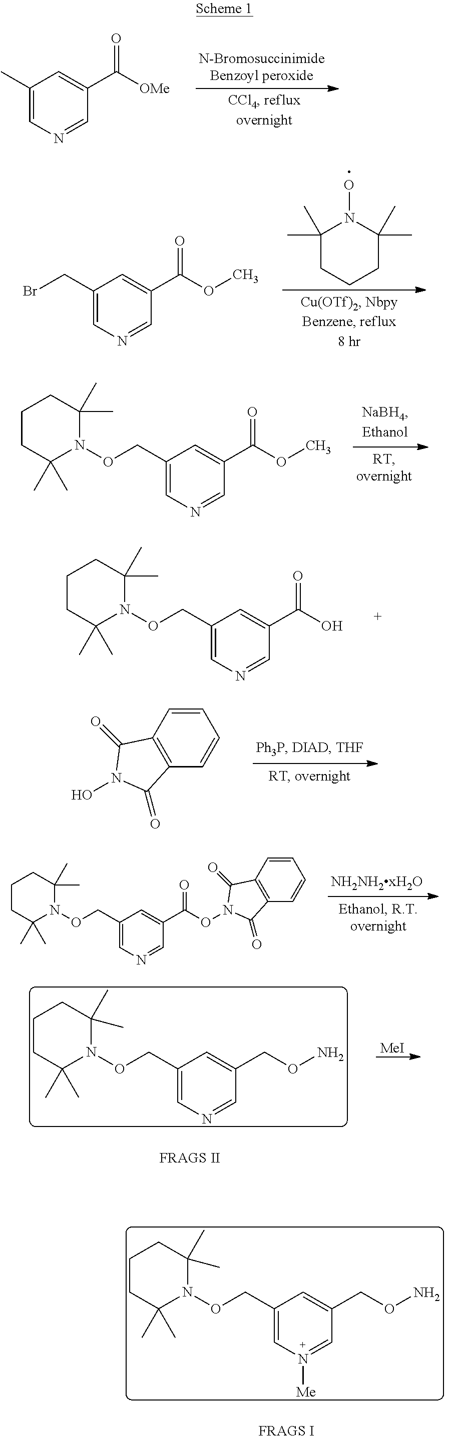

The synthesis strategy for the FRAGS I reagent and FRAGS II reagent is summarized in Scheme 1 above. (Syntheses of reactants shown here in Scheme 1 are described in Example 8.) The FRAGS II reagent synthesis was accomplished by benzylic bromination with N-bromosuccinimide (NBS), coupling with TEMPO, reduction of the ester group, activation of the hydroxyl group, and finally hydrazinolysis of the imide group. For FRAGS I synthesis, methyl iodide (iodomethane) was added to the FRAGS II product. A similar synthesis strategy excluding TEMPO derivatization was employed to synthesize the PRAGS reagent (1), as shown in Scheme 2 below.

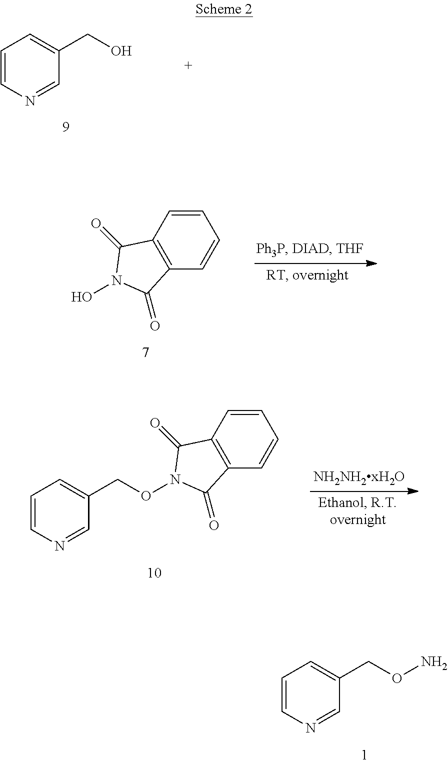 [SMILES: OCc1cccnc1 ; O=C1c2ccccc2C(=O)N1O ; O=C1c2ccccc2C(=O)N1OCc1cccnc1 ; NOCc1cccnc1]

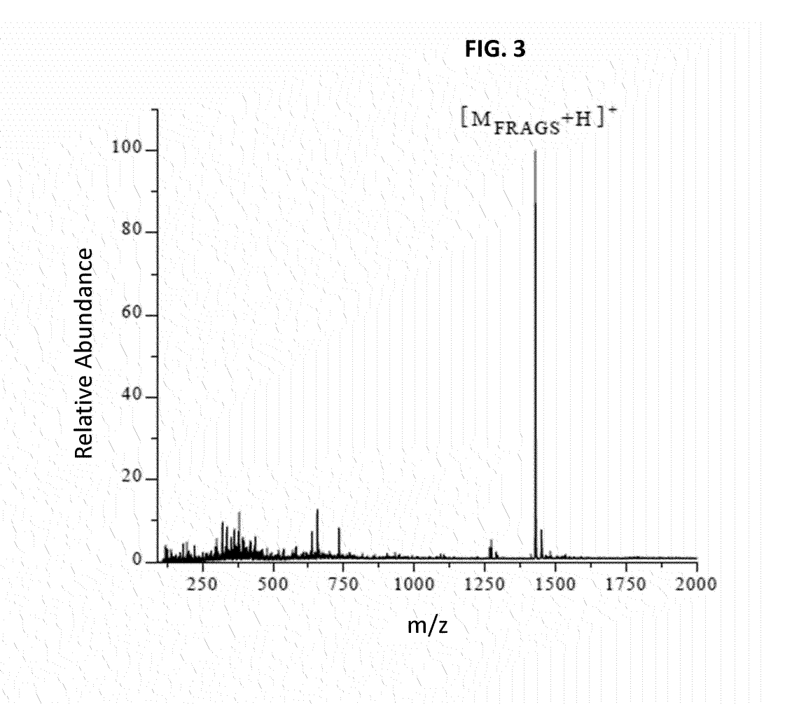

For glycan derivatization, 2 uL of a 20 mM solution of the final product (1 or 2) in acetonitrile (ACN) was mixed with 10 uL of a 1 mM solution of the glycan in H₂O with 1% acetic acid (pH about 4.6). The reaction mixture was allowed to react at 60° C. for 5 hours. After desalting with C18 pipet tips according to the reported protocol, the resulting glycan conjugates were ionized by electrospray ionization (ESI) coupled with an ion trap mass spectrometer (FIG. 3). (Harvey et al., Current protocols in protein science/editorial board, John E. Coligan . . . [et al.]2006, Chapter 12, Unit 12-7, the entire contents of which are herein incorporated by reference.)

### Example 2

### Maltoheptaose

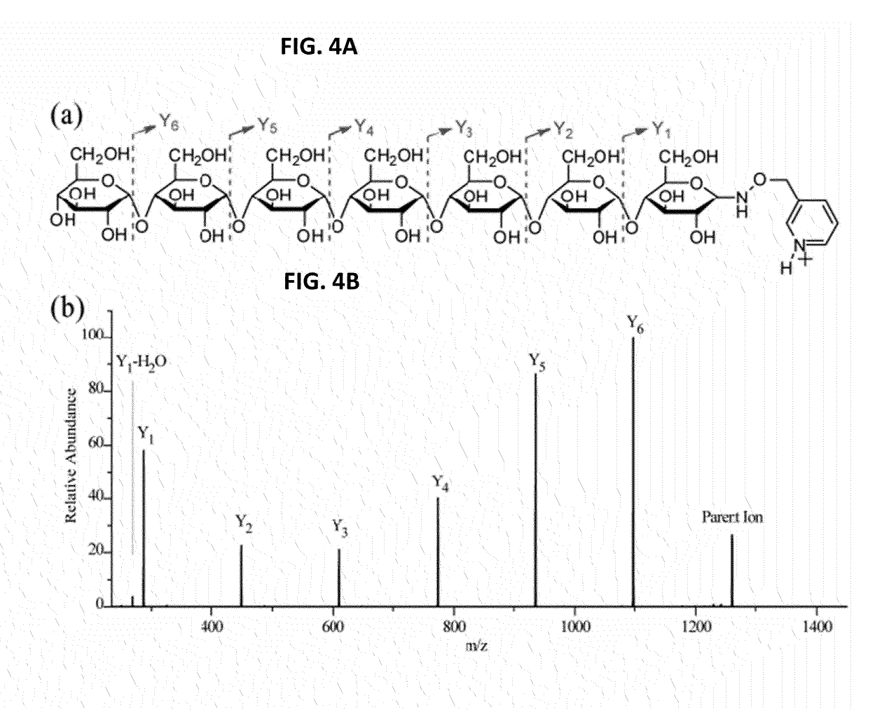

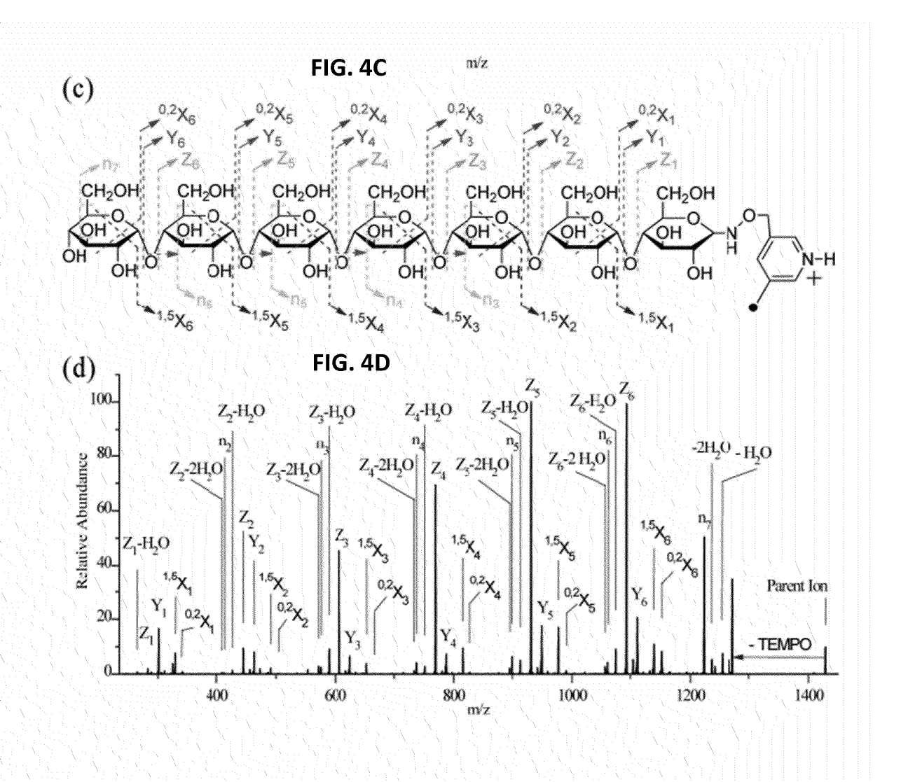

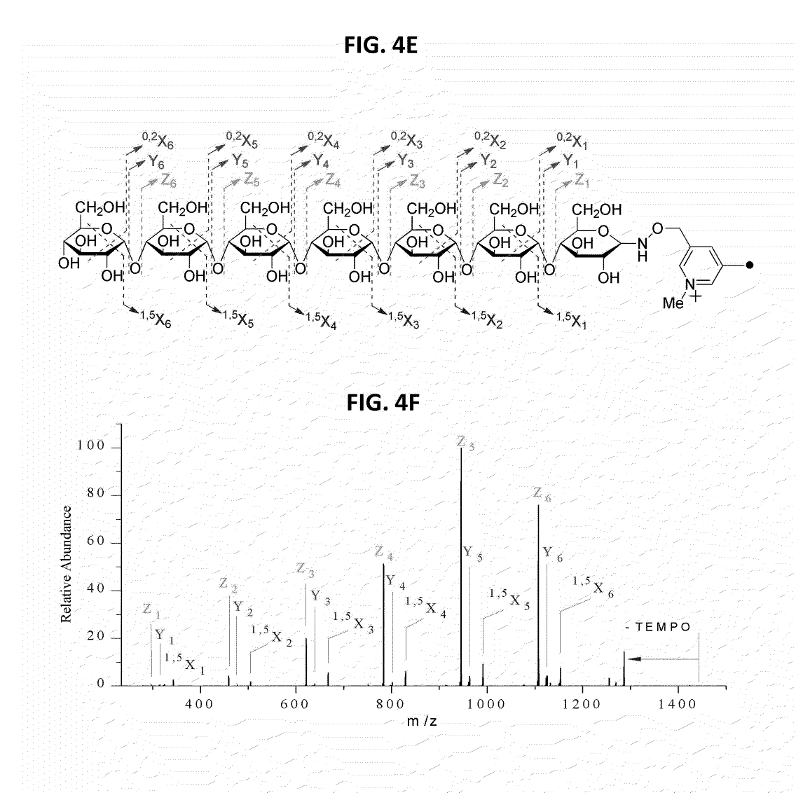

Maltoheptaose presents a linear chain of seven identical glucose subunits (FIGS. 4A-4F). The gas-phase collisional activation of singly-protonated PRAGS-derivatized maltoheptaose generates extensive fragmentation resulting only from C1-O glycosidic bond cleavages, retaining charge on the reducing terminus (Y ions, FIGS. 4A, 4C, 4E). Except for Y₁, probabilities of glycosidic bond cleavage appear to increase with distance from the reducing terminus of the glycan. Enzymatic glycosidic bond hydrolysis takes advantage of acid-base catalysis to achieve selective cleavage of the glycosidic bond. Here, the Y ions are proposed to form via a stepwise mechanism such as postulated in Scheme 3 below.

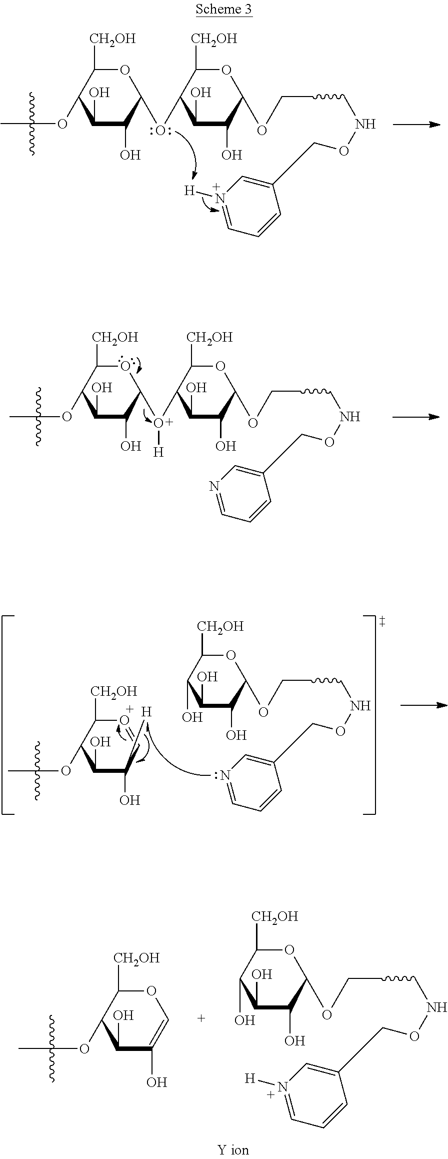

In the first step of Scheme 3, the protonated pyridinium cation functions as a general acid catalyst by protonating the glycosidic oxygen. In the second step, the glycosidic bond is cleaved via the participation of the lone pair of electrons on the endocyclic oxygen to form a transition state resembling an oxocarbenium ion, with the pyridine moiety still in close proximity to the cleaved glycosidic bond. In the final step, this pyridine moiety serves as a base to deprotonate the oxocarbenium ion to form the observed Y ion. The gas phase proton affinity of oxygen atoms in α-D-glycopyranose are calculated to be 204-214 kcal/mol, which is smaller than that of pyridine, 222 kcal/mol. (Stubbs et al., Chem.-Eur. J. 2005, 11, 2651-2659; and Hunter et al., J. Phys. Chem. Ref. Data 1998, 27, 413-656, the entire contents of both of which are herein incorporated by reference.) Alternatively, association of the protonated pyridine with the glycosidic oxygen facilitates a β-hydrogen transfer to oxygen as postulated in Scheme 4 below, which is similar to the reported β-hydrogen transfer of protonated diethylether resulting in loss of ethylene under multiphoton dissociation.

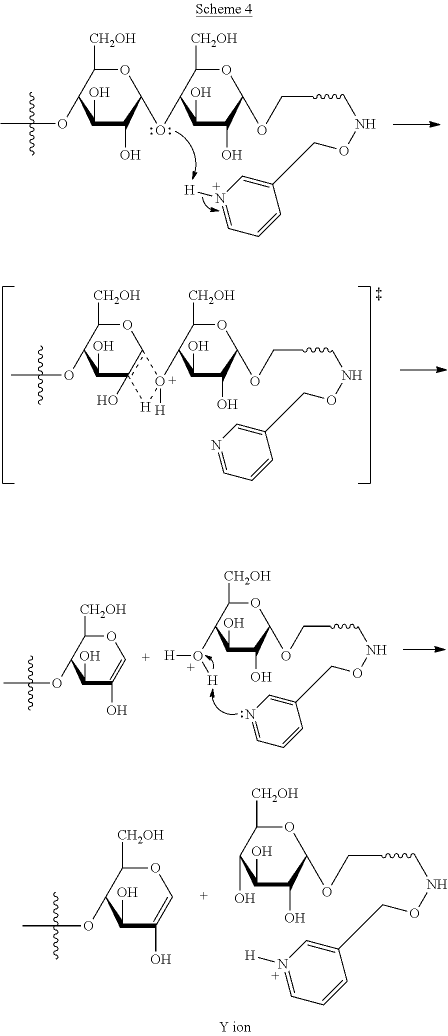

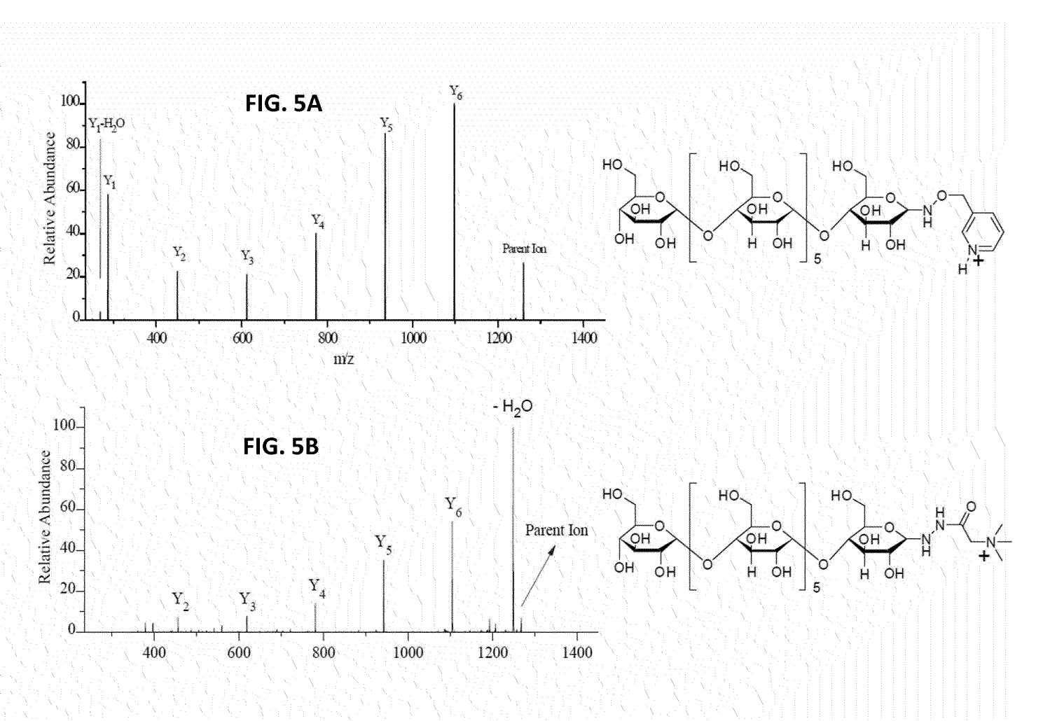

The requirement of acid catalysis involving the labile proton is supported by CID of singly-protonated maltoheptaose derivatized with Girard's T (GT) reagent, a similar aldehyde- and keto-reactive molecule with a fixed-charge quaternary ammonium moiety. In contrast to the protonated charge site of the PRAGS reagent, the inability of this reagent to donate a proton leads to an increase in the CID energy required for a significant extent of fragmentation, and the spectrum is dominated by loss of H₂O instead of glycosidic bond cleavage as shown in FIGS. 5A-5B. The glycosidic bond cleavage in this case is proposed to result from a concerted four-member ring rearrangement, needing energy input to overcome the reaction barrier higher than that required for acid/base catalyzed glycosidic cleavage (Scheme 5).

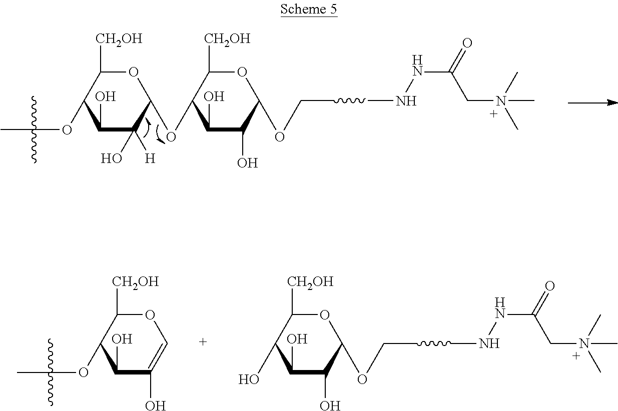

Maltoheptaose was employed as a model for linear glycans as it has been well studied by CID, ECD, and EDD mass spectrometry. (Adamson et al, supra; Zhao et al., J. Am. Soc. Mass Spectrom. 2008, 19, 138-150; Kornacki et al., 2012, 23, 2031-2042; Yu et al., Anal. Chem. 2012, 84, 7487-7494, the entire contents of all of which are herein incorporated by reference.) The peak assignments are unambiguous as only Y ions are observed, providing composition and sequence information for the structural analysis of maltoheptaose. This behavior is different from the reported CID of [M+Metal]⁺, [M_{permethylated}+Na]⁺, [M−H+Cl]²⁻, and [M−2H]²⁻ glycans, wherein not only Y ions but also A, B, C, X, and Z ions are observed. (Lewandrowski et al., Anal. Chem. 2005, 77, 3274-3283; Zhao et al., supra; Kornacki et al., supra; and Adamson et al., supra. the entire contents of all of which are herein incorporated by reference.) Due to the symmetry of maltoheptaose, multiple pairs of isobaric product ions (B and Z, C and Y, as well as A and X ions) make the assignment ambiguous.

Collisional activation of singly-protonated FRAGS II-derivatized maltoheptaose induces not only C1-O glycosidic bond cleavage but also O-Cx glycosidic bond cleavage and cross-ring cleavages. Many more free radical driven fragmentation pathways in the gas-phase are observed compared with that observed previously in solution. (Duan et al., Glycobiology, 2011, 21: 401-409, the entire contents of which are herein incorporated by reference.) A series of abundant and systematic dissociation patterns including ^{0,2}X, ^{1,5} X, Z, and n ions are observed and proposed to be driven by hydrogen abstraction followed by rearrangement. As the Z, ^{1,5}X, ^{0,2}X, and n ions are not observed in the CID spectrum of singly-protonated PRAGS-derivatized maltoheptaose, they are proposed to occur via free radical driven mechanisms. In the first step of Z ion formation, the nascent free radical, the carbon-centered radical formed on the FRAGS II reagent, abstracts a hydrogen atom from C5 to generate a carbon-centered radical at this site. In the second step, the resulting radical promotes homolytic cleavage of the glycosidic bond via formation of a double bond between C4 and C5 as shown in Scheme 3.

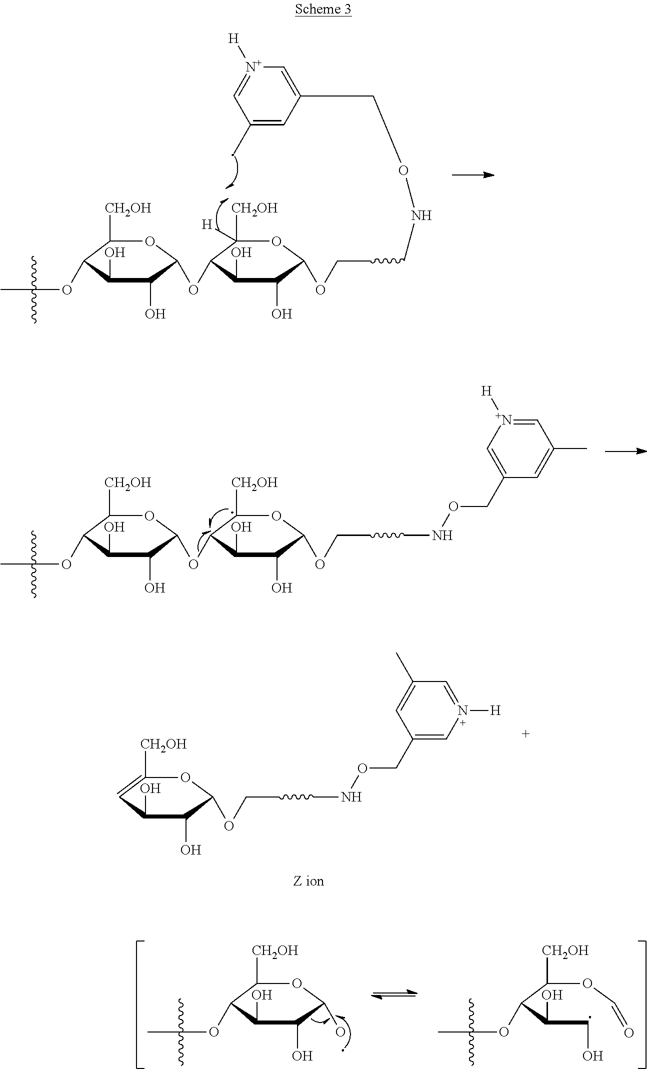

An alternative mechanism involving hydrogen abstraction from non-reducing terminus C′1-H′1 (anormeric) followed by O—C4 homolytic cleavage and loss of C5-H5 radical is also possible as shown in Scheme 4.

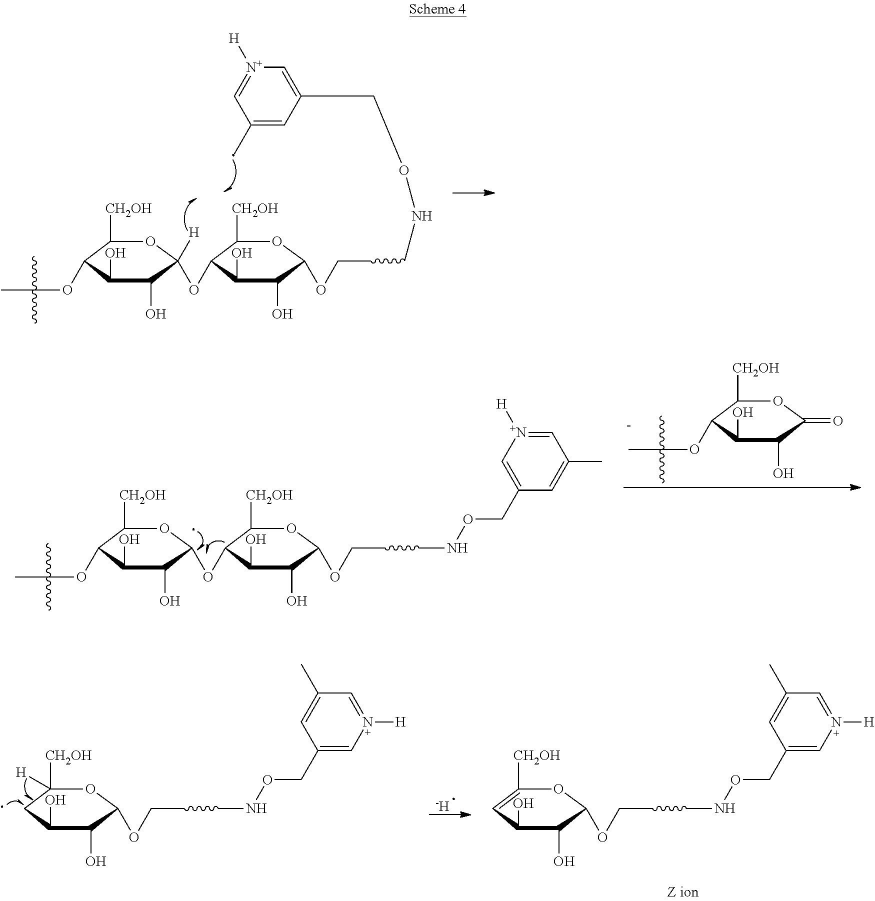

Similar to the formation of Z ions, the formation of ^{1,5}X ions as shown in Scheme 5, ^{0,2}X as shown in Scheme 6, and n ions as shown in Scheme 7, is initiated by hydrogen abstraction by the nascent free radical followed by β-bond cleavages.

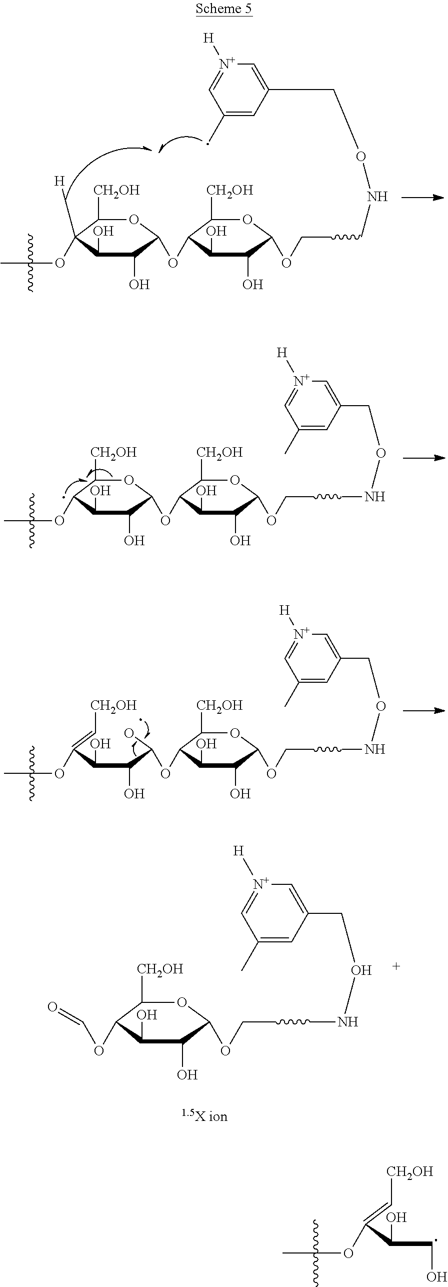

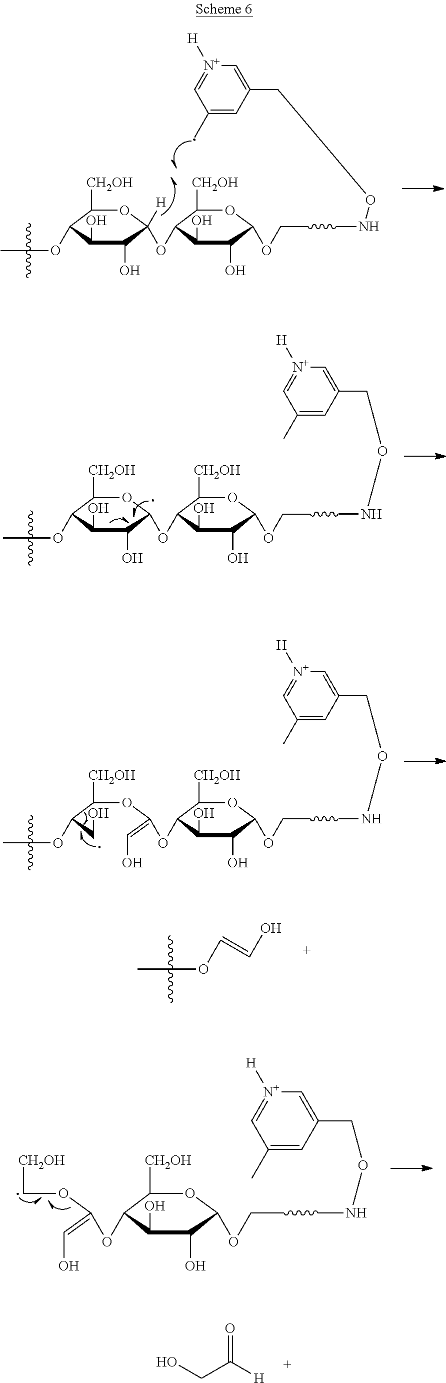

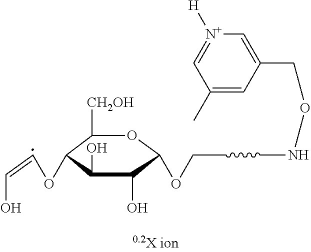

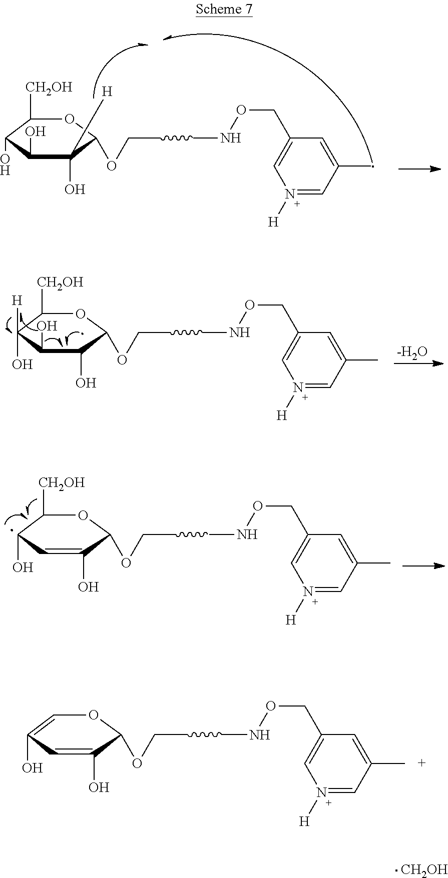

Compared with CID of singly-protonated PRAGS-derivatized maltoheptaose, CID of singly-protonated FRAGS II-derivatized maltoheptaose provides much more structural information (FIGS. 4A-4D). All the Y-type fragmentation generated from CID of singly-protonated PRAGS-derivatized maltoheptaose is observed in the CID spectrum of singly-protonated FRAGS II-derivatized maltoheptaose. The observed product ions are sufficient for the comprehensive structural analysis of maltoheptaose. Systematic glycosidic bond cleavages (Y and Z ions) provide composition and sequence information while the cross-ring cleavages (^{0,2}X and ^{1,5}X ions) and loss of CH₅O₂ (n ion) provide linkage information. For further comparison, the fragmentation pattern and CID spectrum of FRAGS I-derivatized maltohelptaose (FIGS. 4E-4F) shows a decrease in the number of product ions, thereby decreasing the complexity of the spectra. Employing the FRAGS I, FRAGS II and PRAGS reagents, the majority of the product ions retain charge on the reducing terminus, simplifying the analysis of CID spectra. The systematic cross-ring and glycosidic bond cleavages observed with high fragmentation efficiency using the FRAGS II reagent provides the expectation that unknown glycan structures can be inferred from their fragmentation patterns.

### Example 3

### Disaccharide Isomers

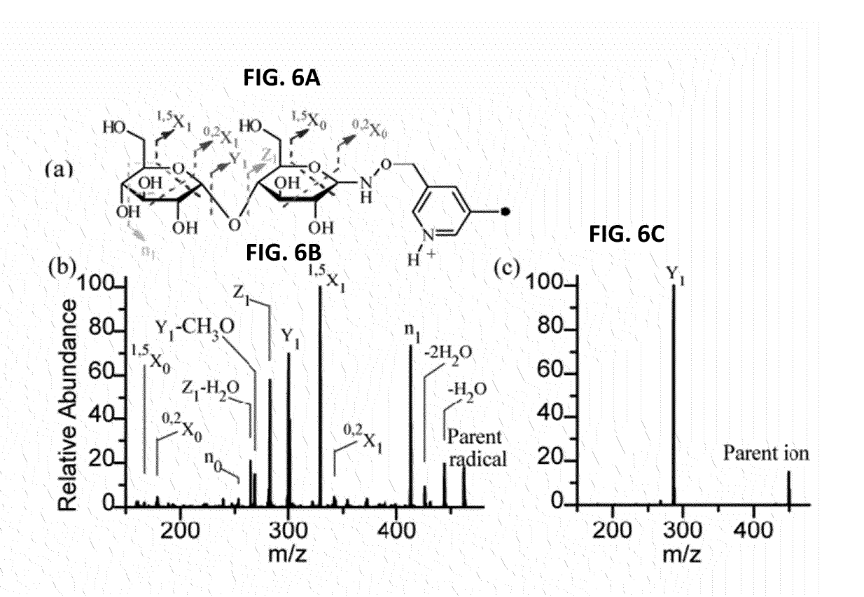

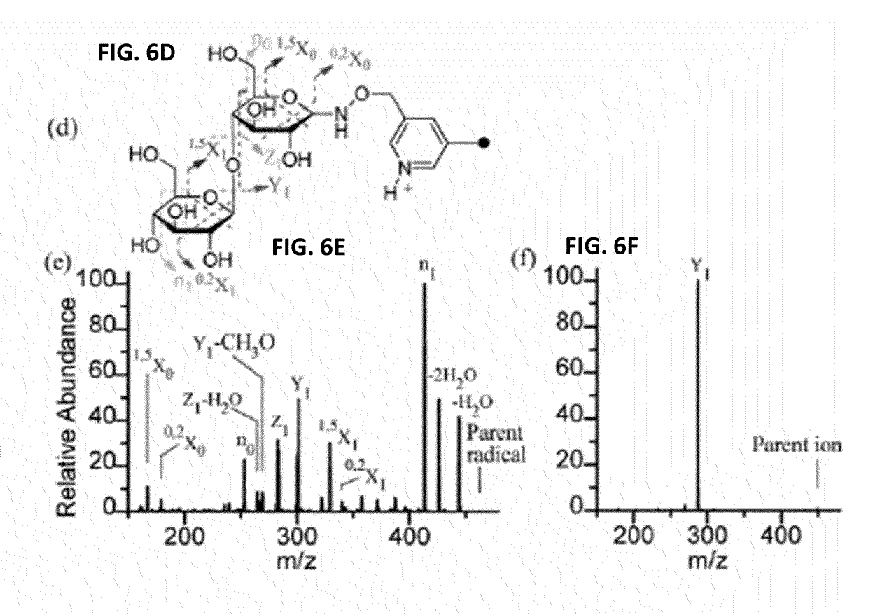

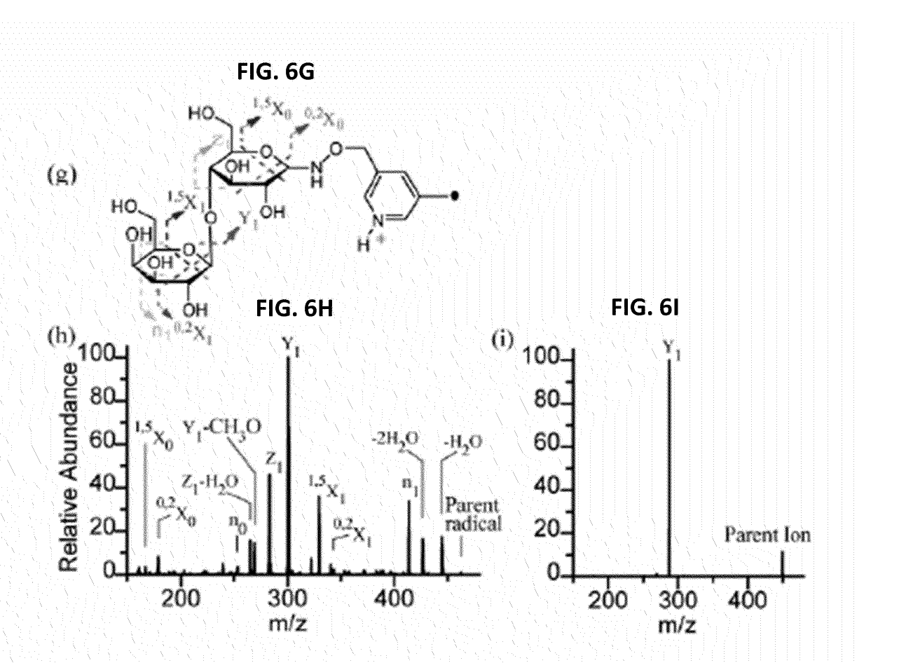

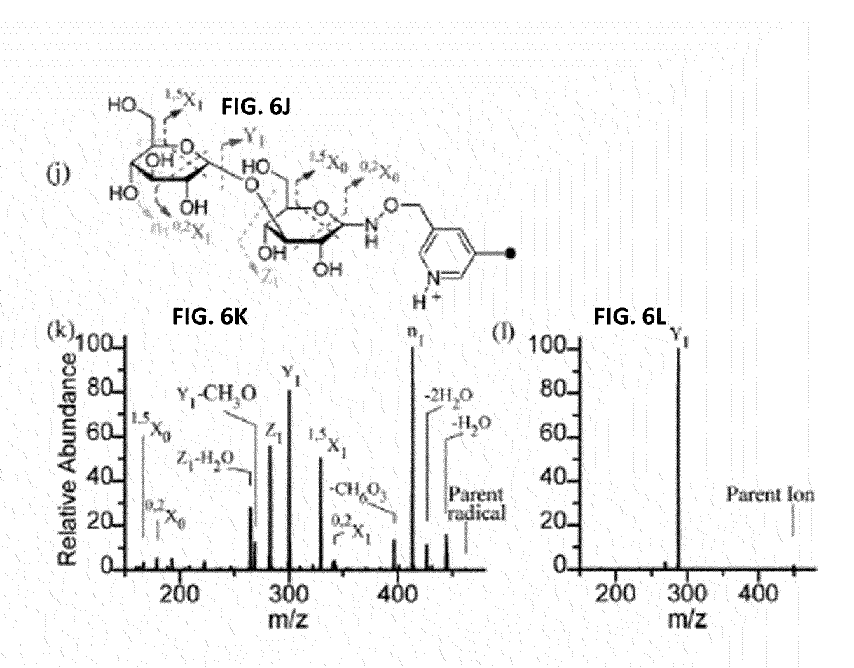

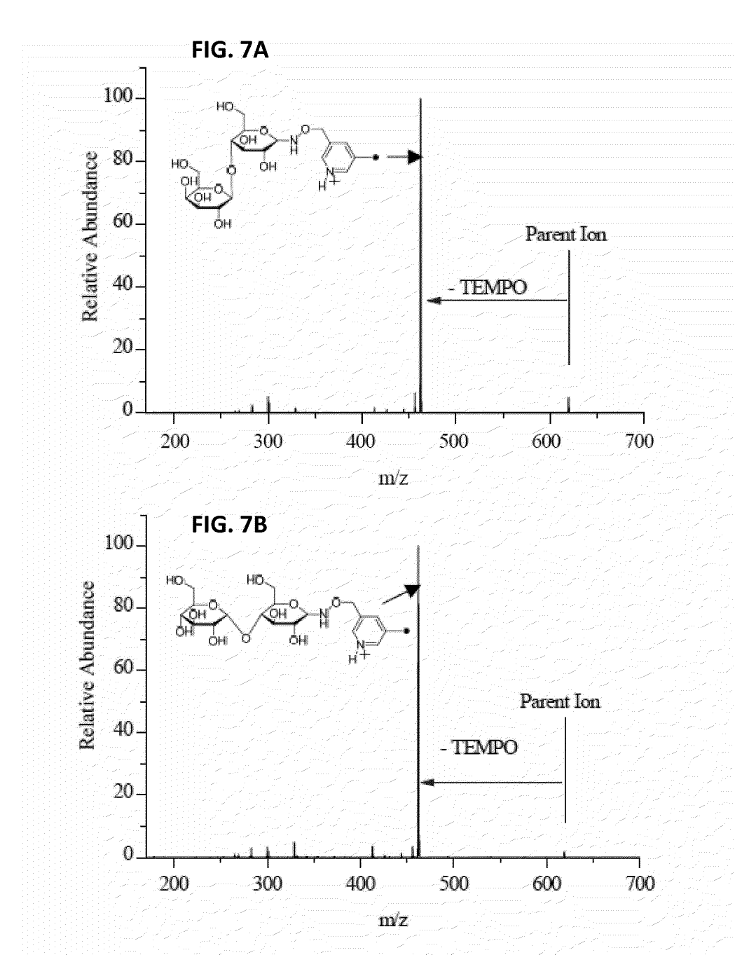

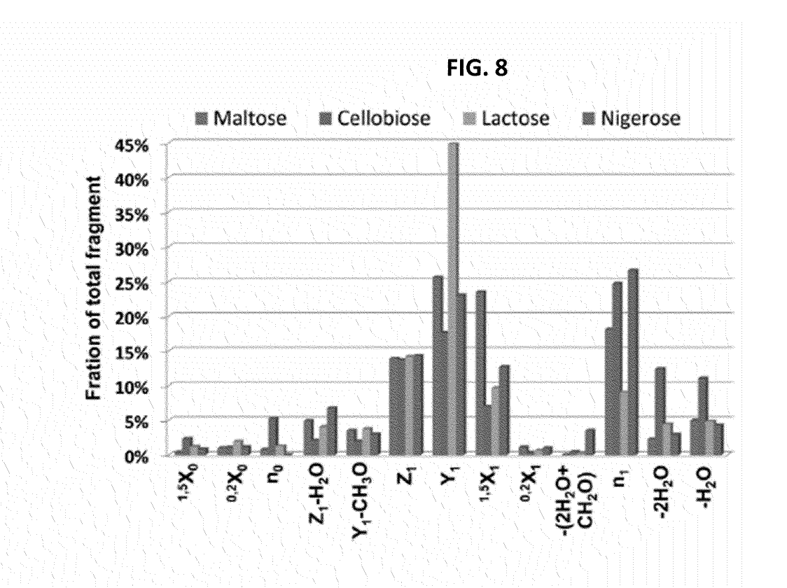

To assess the ability of the PRAGS and FRAGS I reagents to differentiate isobaric glycan structures, the dissociation of several disaccharide isomers was examined. Maltose is a glycan that consists of two glucose bonded through an α1-4 linkage, whereas cellobiose is an isobaric glycan that consists of two glucose bonded through an β1-4 linkage. Lactose differs from cellobiose only in the stereochemistry of C4 in the non-reducing terminus glycan subunit, whereas nigerose differs from maltose and cellobiose only in the linkage type. Collisional activation of these four singly-protonated PRAGS-derivatized disaccharides generates exclusively C1-O glycosidic bond cleavage (forming a Y ion in FIGS. 6B, 6E, 6H, and 6K), with no significant difference in the mass spectra of the four structural isomers. However, with the FRAGS-derivatized glycans, structures for which are shown in FIG. 3, collisional activation of [M+H-TEMPO]⁺ ions of these four disaccharides generates more extensive fragmentation as shown in FIGS. 6A, 6D, 6G, and 6J, including glycosidic bond cleavage (Y and Z ions), cross-ring cleavage (^{1,5}X and ^{0,2}X), and —CH₅O₂ (most likely H₂O+HOCH₂.) cleavage (n ion). The [M+H-TEMPO]⁺ ions of the disaccharides are generated by CID of the corresponding singly-protonated FRAGS-derivatized glycans as shown in FIGS. 7A, 7B. While the FRAGS CID spectra of the four isomers generate the same product ions, the relative intensities are significantly different. In order to clearly see how the FRAGS reagent differentiates the four disaccharide isomers, the branching ratios (fraction of total fragment yield) of each sugar are compared (FIG. 8). Significant differences are observed in fragment yields of Y₁, ^{1,5}X₁, —CH₆O₃ (e.g., CH₂O+2H₂O), n₀, and n₁. The ^{1,5}X₁, ion is the base peak (most abundant fragment) for maltose, the n₁ ion is the base peak for cellobiose, and the Y₁ ion is the base peak for lactose. The significant decrease in intensity of the n₁ ion for lactose is attributed to the presence of a cis-diol on C3 and C4, according to the general mechanism postulated for the formation of n ion as shown in Scheme 7. The relatively high intensity of the ^{1,5}X₁ ion for maltose may be due to the fact that the hydrogen on C4′ (non-reducing subunit) of maltose is more accessible than in the other isomers. Although the n₁ ion is the base peak for both nigerose and cellobiose, the relative abundances of the Y₁ and —CH₆O₃ ions are higher for nigerose. The neutral loss of CH₆O₃ is proposed to occur via hydrogen abstraction by the nascent free radical followed by rearrangement, as shown in Scheme 8.

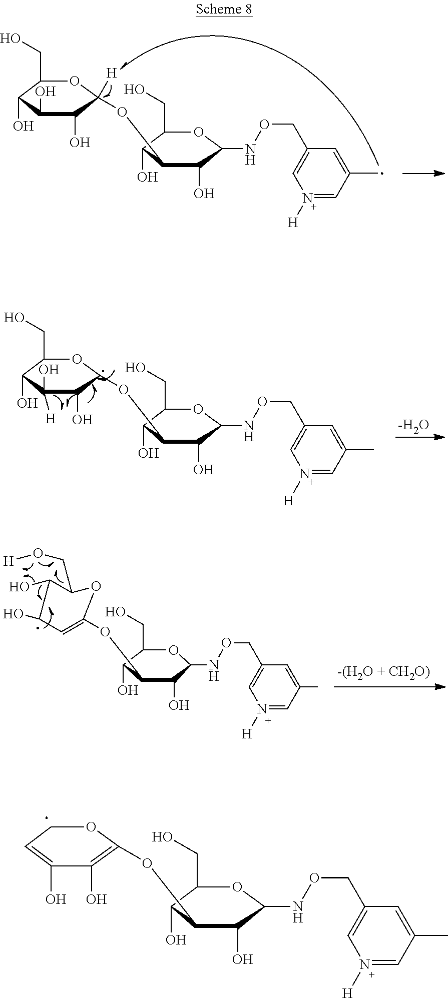

The relatively high intensity of the —CH₆O₃ ion for nigerose compared to the other three isomers may be rationalized by the fact that the hydrogen on C1′ (non-reducing subunit) is highly accessible to the nascent free radical. Clearly, the no ion of cellobiose is significantly more abundant compared to the other three disaccharide isomers. Overall, the difference in fragment ion abundances in the FRAGS CID spectra allows the glycan isomers to be readily distinguished.

### Example 4

### Lacto-N-dificohexaose II (LNDFH II)

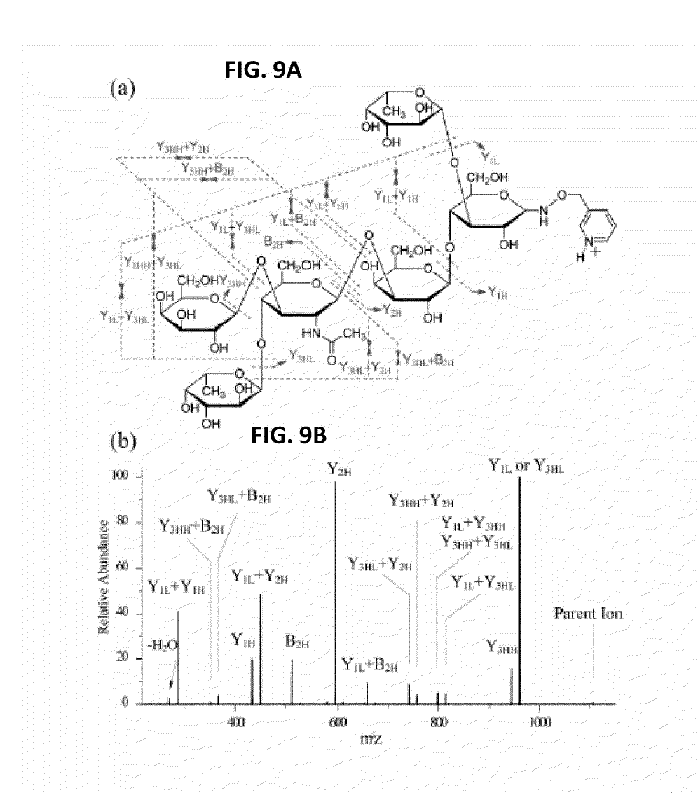

Two complex branched glycans, Lewis-Y tetrasaccharide and LNDFH II, were also examined with the new reagents. LNDFH II is employed as a highly branched model glycan, having a reducing terminus in the center and a branch on the N-acetylglucosamine unit, to assess the ability of FRAGS and PRAGS reagents to analyze more complicated glycan structures. Collisional activation of singly-protonated PRAGS-derivatized LNDFH II (FIGS. 9A, 9B) mainly generates not only systematic Y ions but also Y+Y ions. The Y+Y ions result from a pair of C1-O glycosidic bond cleavages. As with the simple glycans discussed above, the PRAGS reagent systematically deconstructs the glycan, revealing subunit connectivity. This can be illustrated in the deconstruction (DECON) diagram, which serves to visually summarize the MSⁿ results, shown for LNDFH II in FIG. 10. The sequence, composition and branching of LNDFH II can be discerned from this diagram. Three pairs of product ions, Y_{3HH} (m/z 960.6) and Y_{3HL} (m/Z 944.6), Y_{1L}+Y_{3HH} (m/z 814.5) and Y_{1L}+Y_{3HL} (m/z 798.5), as well as Y_{1L}+Y_{2H} (m/z 499.5) and Y_{1H} (m/z 433.4), can be employed to infer the existence of two branches in the glycan structure. Moreover, Y_{1L}+Y_{2H} and Y_{1H} can be employed to elucidate the site of the reducing terminus of LNDFH II. It is noted that the Y_{3HL}+Y_{2H} and Y_{3HH}+Y_{2H} ions result from the internal loss of N-acetylglucosamine, and Y_{1L}+B_{2H} is formed by the migration of fucose. Furthermore, the generation of B_{2H} (m/z 512.5), Y_{3HL}+B_{2H} (m/z 366.3), and Y_{3HH}+B_{2H} (m/z 350.3) ions provides information on the location of N-acetylated saccharide units, even though they make the spectrum more complicated. This can be rationalized by considering the proton affinity of pyridine (222 kcal/mol) and an amide (N-methylacetamide, 212 kcal/mol), which makes it possible to protonate the N-acetyl group and therefore to generate B ions, even though the relative abundance is low compared with that of Y ions (Hunter et al., J. Phys. Chem. Ref Data 1998, 27, 413-656, the entire contents of which are herein incorporated by reference).

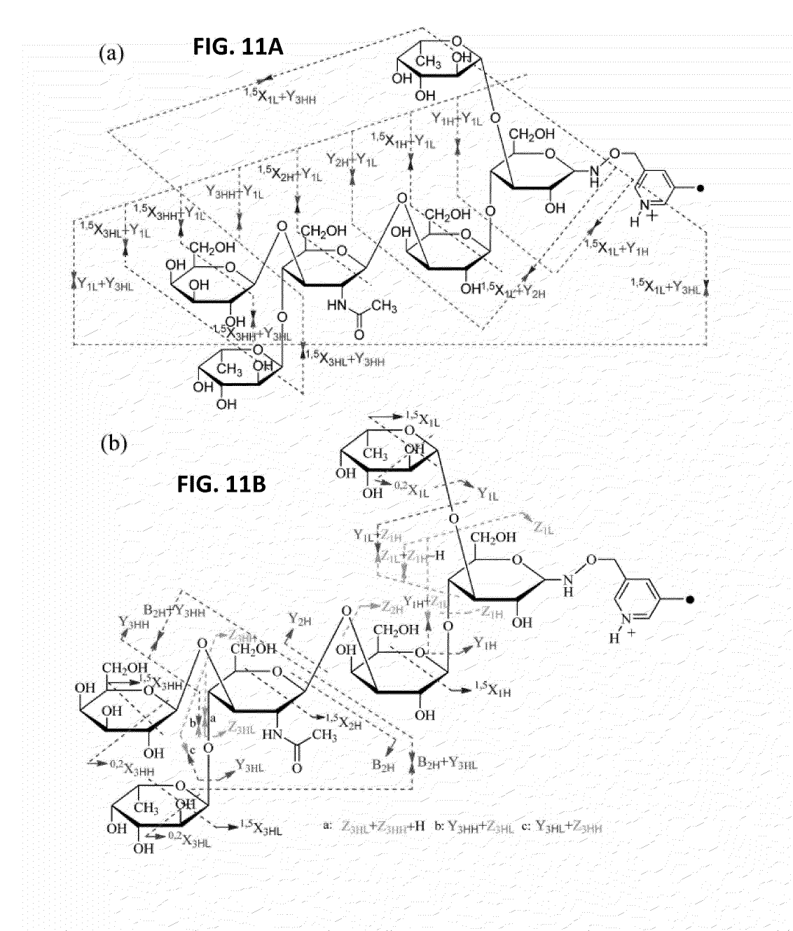

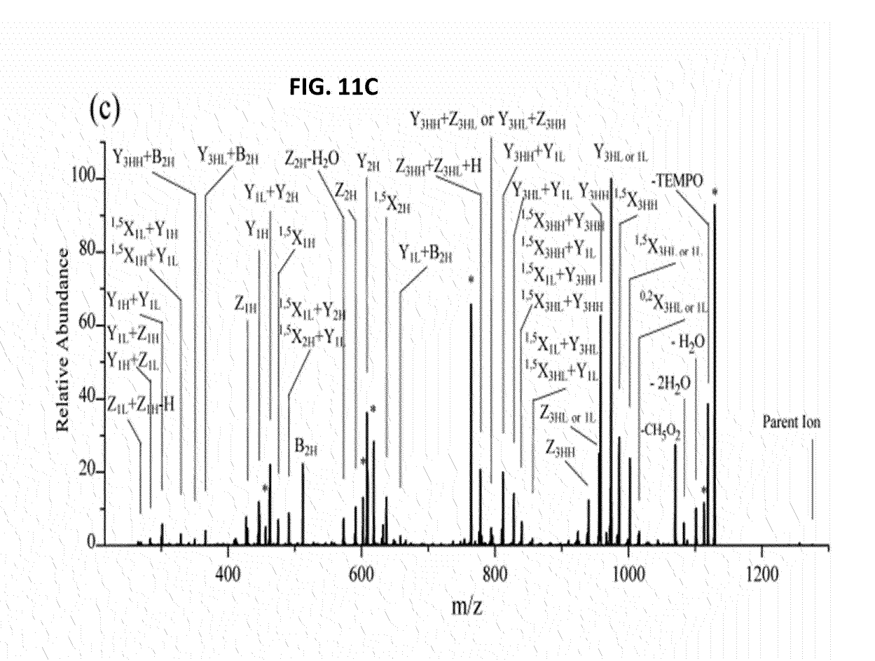

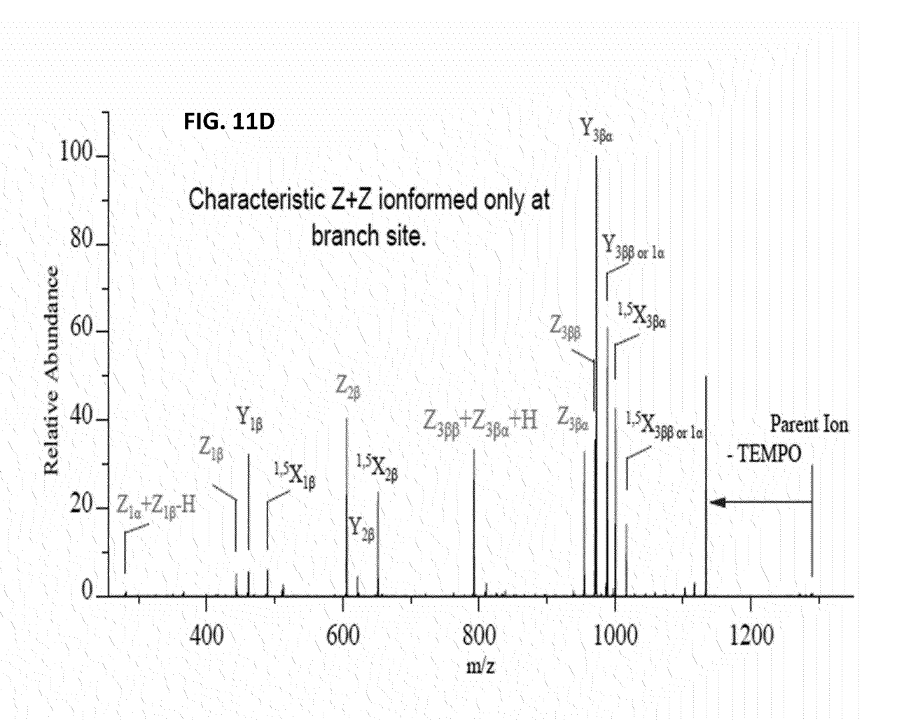

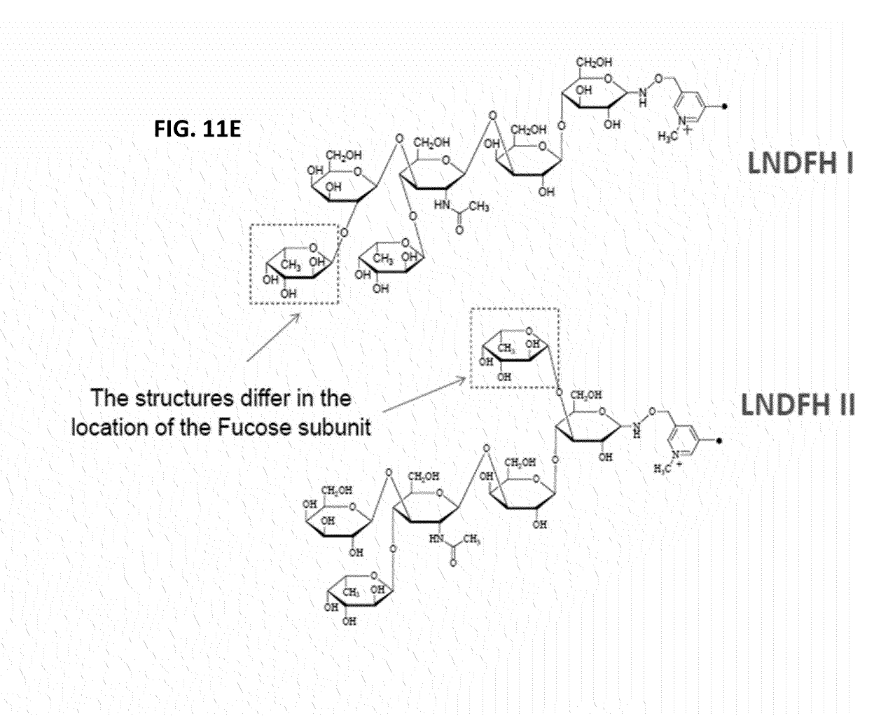

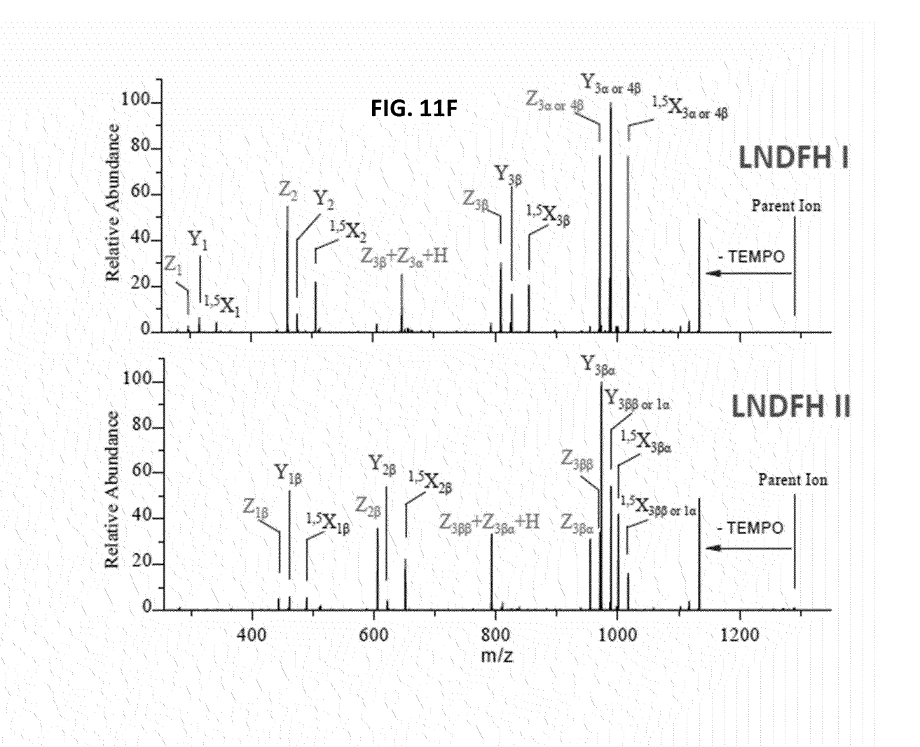

All the fragmentation patterns generated from CID of singly-protonated PRAGS-derivatized LNDFH II can also be found in the CID spectrum of singly-protonated FRAGS II-derivatized LNDFH II (FIGS. 11A-11C). In addition, the generation of systematic series of Y+^{1,5}X ions can be employed to illustrate the existence of the two branch sites in LNDFH II. Four more characteristic ions, Z_{1H}+Z_{1L}−H (m/z 265.1), Z_{1H}+Y_{1L} (or Z_{1L}+Y_{1H}, m/z 283.1), Z_{3HH}+Z_{3HL}+H (m/z 778.3), and Z_{3HH}+Y_{3HL} or Z_{3HL}+Y_{3HH}(m/z 794.3) also provide direct evidence for the existence of the two branch structures. The Z_{1H}+Z_{1L}−H ion can be employed to infer the site of the reducing terminus. By comparison, the fragmentation pattern generated from CID of FRAGS I-derivatized LNDFH II is shown in FIG. 11D. Furthermore, FRAGS I-derivatized LNDFH I was fragmented and analyzed compared to LNDFH I. The structural differences are shown in FIG. 11E, and the aligned CID spectra are shown in FIG. 11F. These results for the analysis of the highly branched hexasaccharide LNDFH II employing the PRAGS and FRAGS I and FRAGS II reagents reveal the utility of these reagents for unraveling many of the structural features of highly branched glycans. In addition, the generation of B and B+Y ions upon CID of singly-protonated LNDFH II can be employed to indicate the existence and substitution site of an N-acetylated glycan unit.

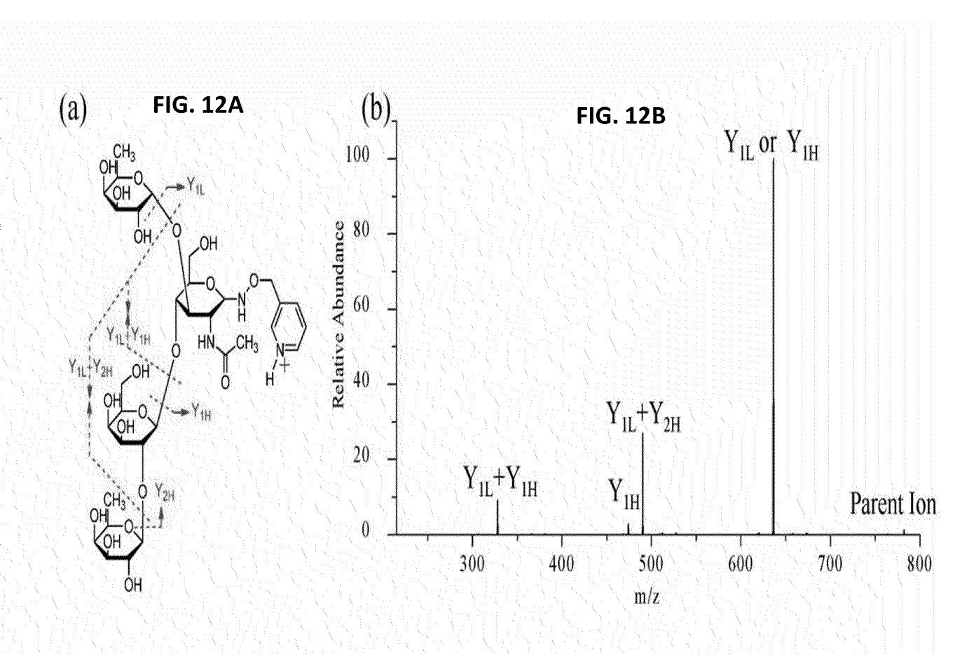

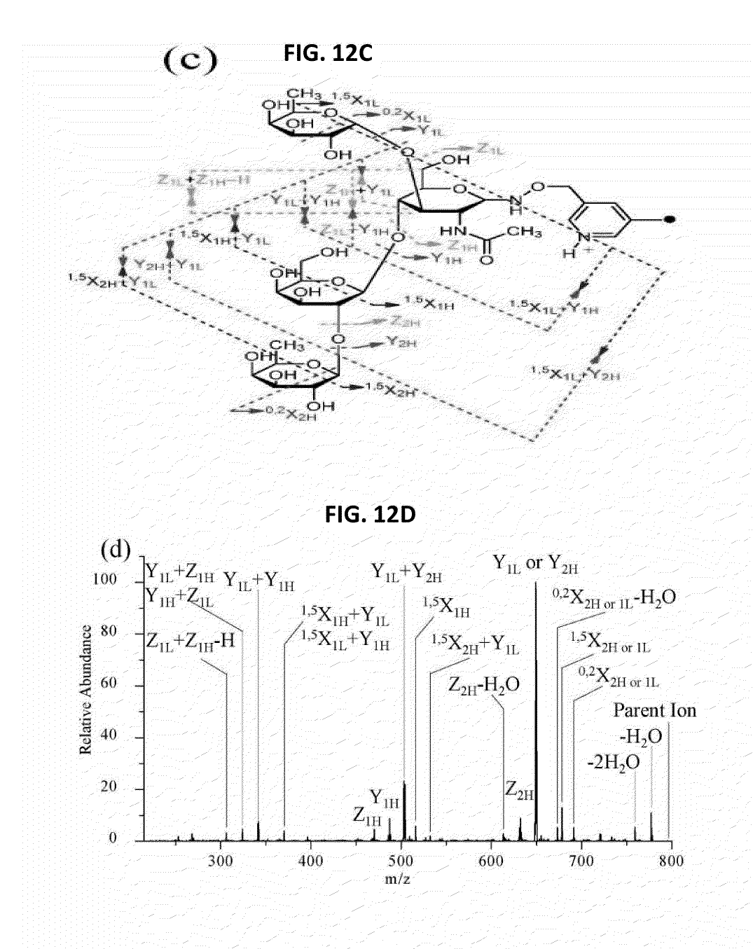

Lewis-Y tetrasaccharide has an analogous structural feature, and the location of the reducing terminus can be similarly inferred from the related fragmentation patterns (FIGS. 12A-12D). A suggested mechanism for the formation of Z_{1H}+Z_{1L}−H as shown in Scheme 9.

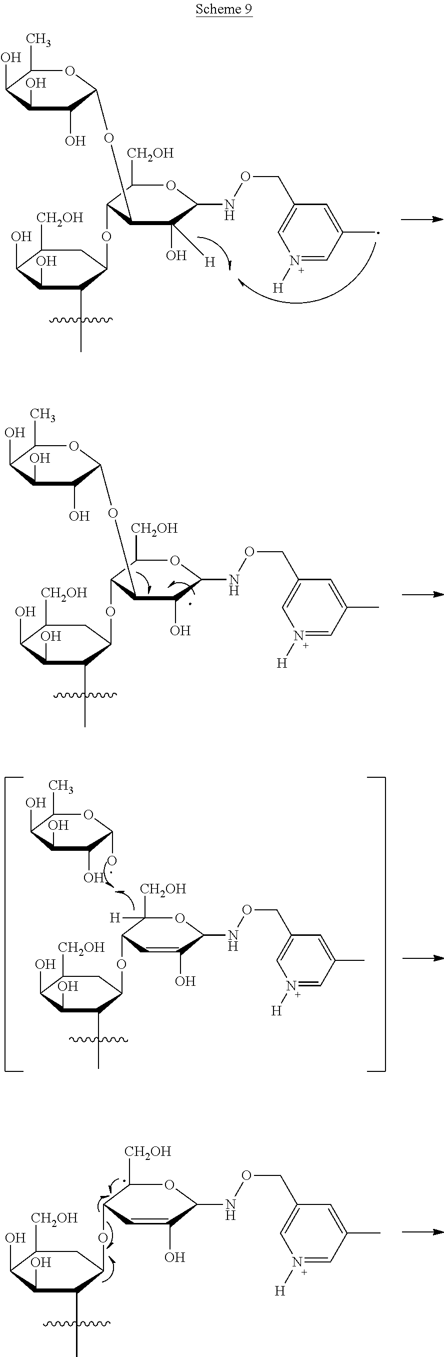

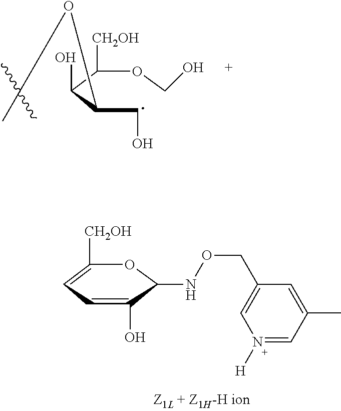

The proposed mechanism of formation of the Z_{3HH}+Z_{3HL}+H ion is shown in Scheme 10.

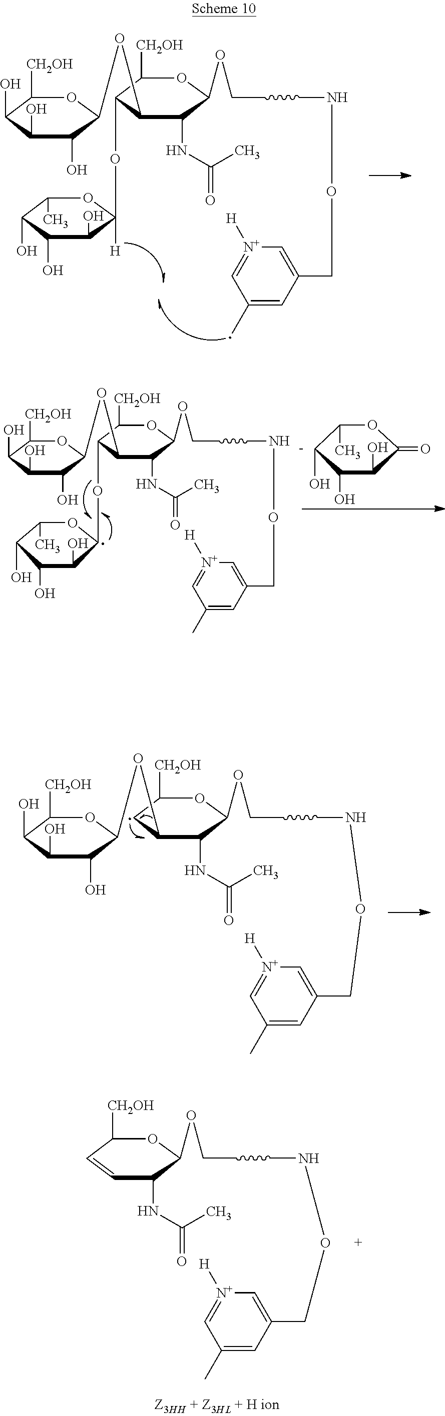

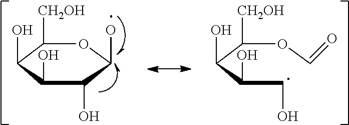

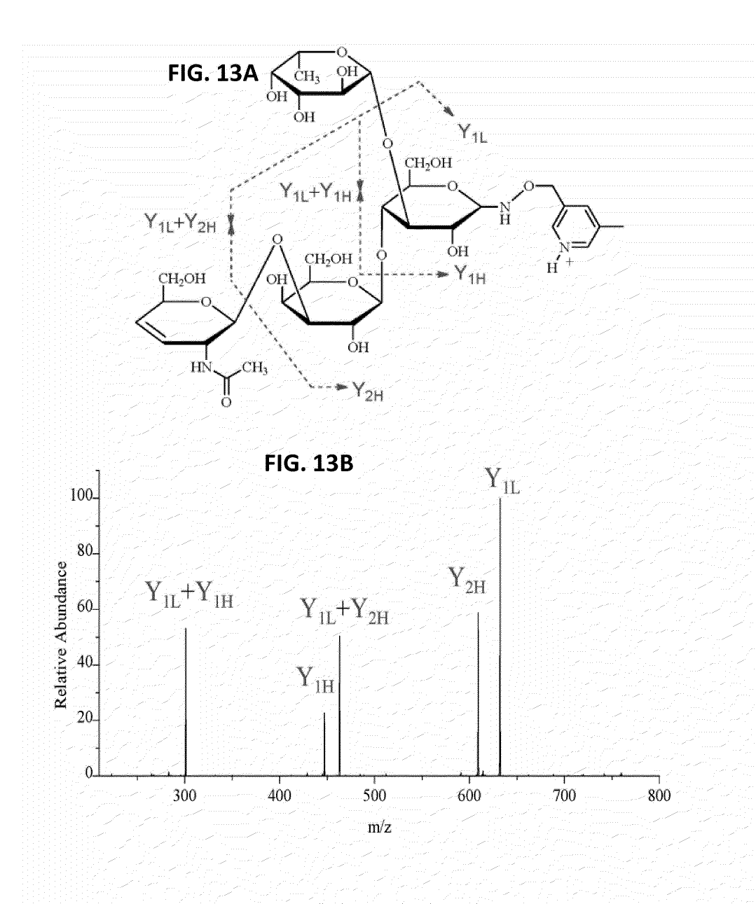

The nascent free radical abstracts a hydrogen atom from C1 of one of the branched glycan units. The resulting carbon-centered radical drives the cleavage of the glycosidic bond and formation of a radical on C4 of the N-acetylglucosamine unit, followed by the formation of a double bond between C3 and C4, and finally a second glycosidic bond cleavage to generate the Z_{3HH}+Z_{3HL}+H ion (Scheme 10). The structure of the Z_{3HH}+Z_{3HL}+H ion is confirmed by CID of this characteristic ion as shown in FIGS. 13A, 13B.

### Example 5

### Isolation of Glycan Using Resin-Supported FRAGS II

As depicted below in Scheme 11, resin (Thiopropyl-Sepharose® 6B) is coupled to the FRAGS II reagent.

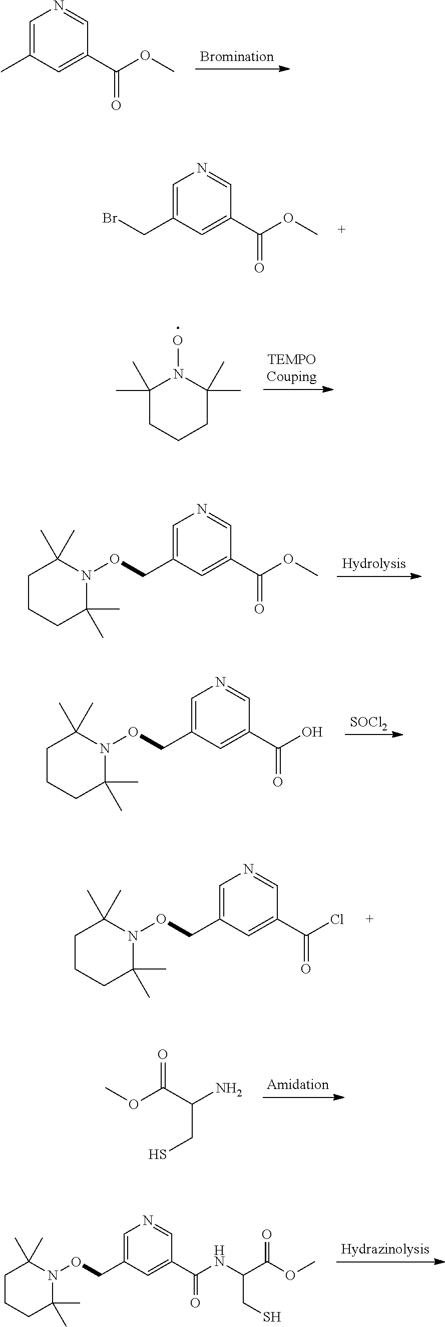

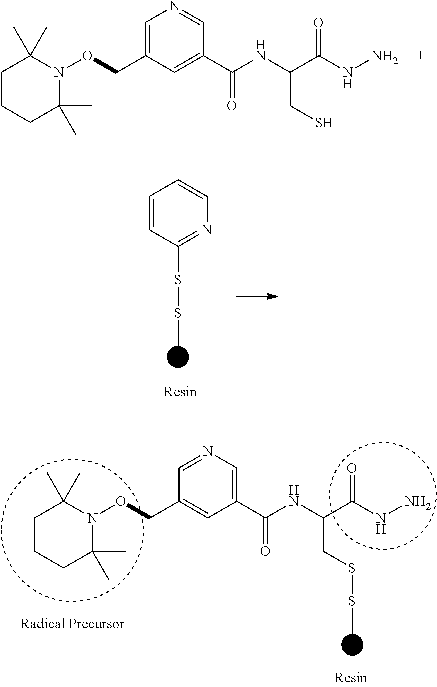

The synthesis strategy for the Resin-Supported FRAGS II reagent is summarized in Scheme 11 above. The Resin-Supported FRAGS II reagent synthesis was accomplished by benzylic bromination with N-bromosuccinimide (NBS), coupling with TEMPO, hydrolysis of the ester group, activation of the carboxylic acid group, amidation, hydrazinolysis, and finally coupling with Thiopropyl Sepharose® 6B resin. (1 mL, 30 μmol) of resin was washed with deionized water. After the final step, the Thiopropyl Sepharose® 6B resin was suspended in 50% methanol. Five equivalents of the FRAGS II reagent (shown after hydrazinolysis step in Scheme 11) was added to the suspension of the Thiopropyl Sepharose® 6B resin. The reagent and resin suspension was placed on a rocking incubator at room temperature (RT) overnight. After incubation, the resins were washed with 50% methanol, followed by water and 20% ethanol. The resulting resins were stored in 20% ethanol at 4° C. prior to use.

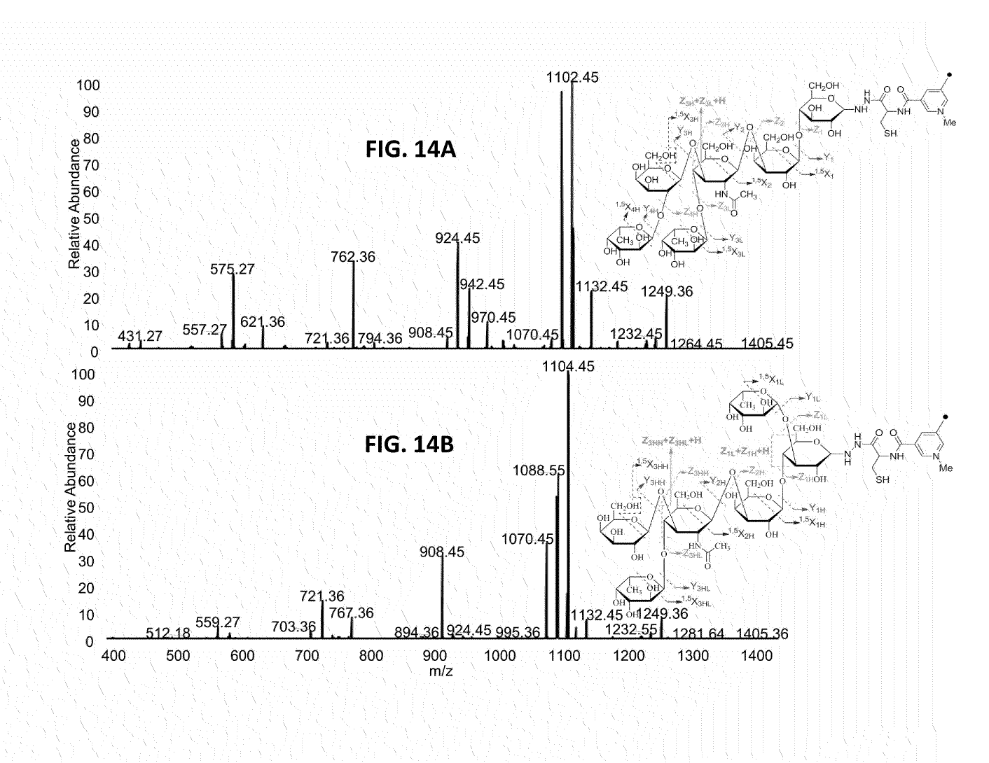

The RS-FRAGS II reagent was coupled with LNFDI and LNFDII. The RS-FRAGS II-derivatized LNFDI and RS-FRAGS II-derivatized LNFDII were treated with dithiothreitol (DTT) to remove the resin support and then subject to ESI and CID by mass spectrometry. The CID spectra for LNFDI and LNFDII using the RS-FRAGS II reagents are shown FIGS. 14A and 14B, respectively.

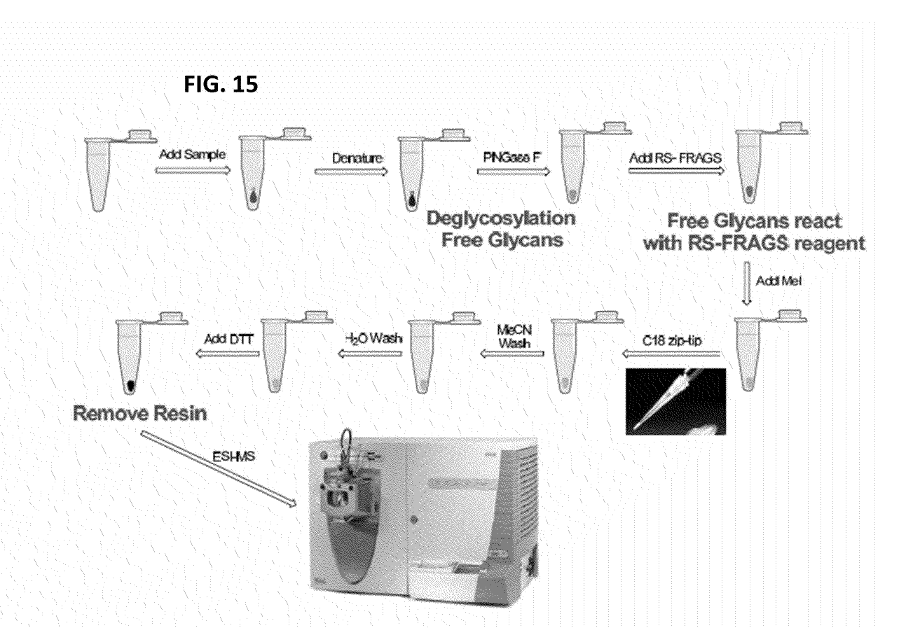

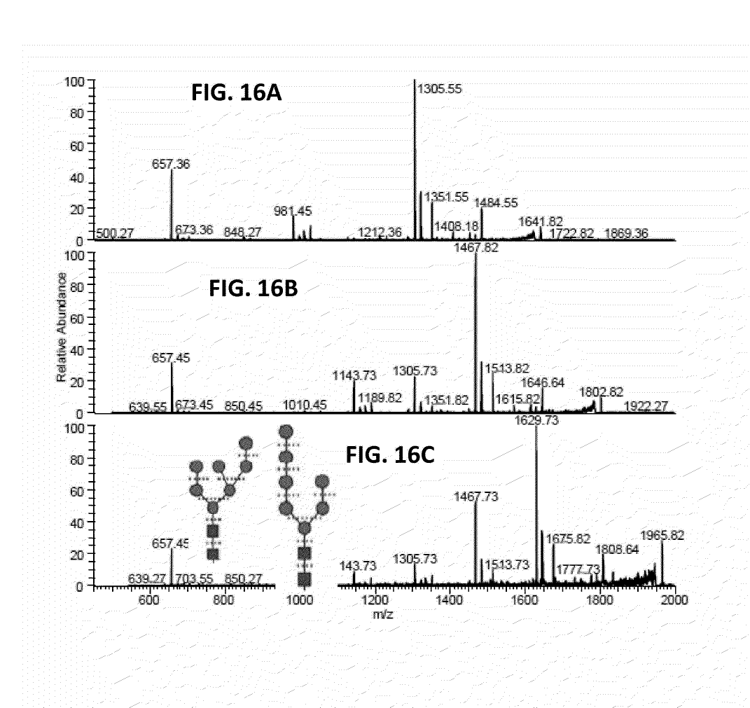

Using the RS-FRAGS II reagent, the glycoprotein ribonuclease B (RNase B) was analyzed following the protocol shown schematically in FIG. 15. In brief, RNase B was treated with PNGase F to release the N-glycans. The RS-FRAGS II reagent was added to derivatize the N-glycans. The reaction mixture was washed with acetonitrile and water sequentially. The resin was removed by adding dithiothreitol (DTT) and finally the derivatized glycans were analyzed by ESI-MS/MS. The CID spectra for the N-glycans isolated from RNase B using RS-FRAGS II are shown in FIGS. 16A-16C. Systematic fragmentation patters (^{1,5}X, Y, and Z) were clearly observed. Moreover, the first branch site from can be easily identified.

### Example 6

### Calculations

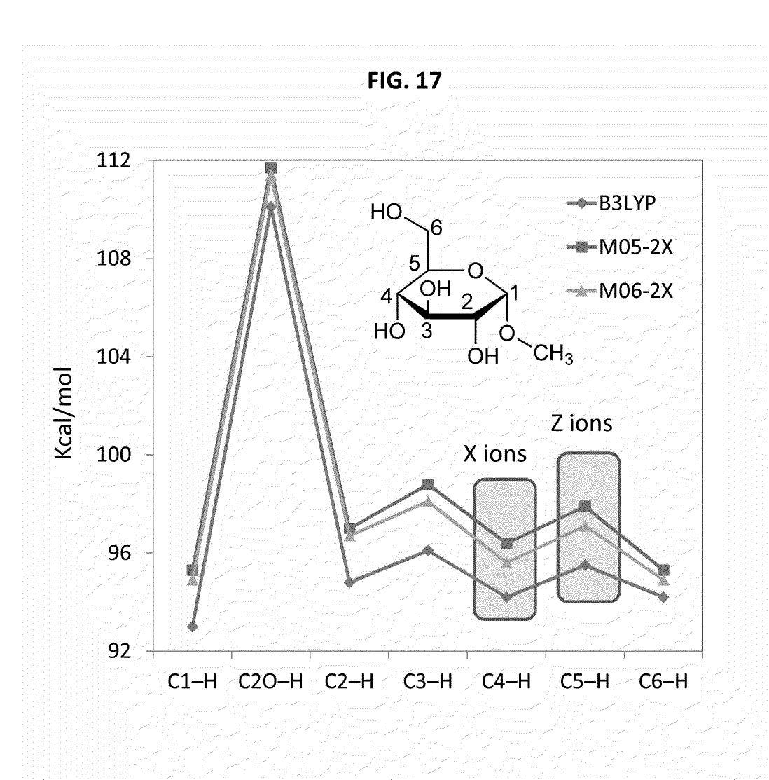

To gain further insight into the hydrogen atom abstraction processes observed with the FRAGS reagent, C—H and O—H bond dissociation enthalpies (BDEs) of α-1-O-methyl-D-glucopyranose were calculated as described in Example 1. These are displayed graphically in FIG. 17. In agreement with the proposed mechanisms and previous literature, the BDEs of O—H bonds are significantly higher than those of C—H bonds. In all cases, the C—H BDEs for the glycan fall within a narrow range and indicate a slightly endothermic reaction for hydrogen atom abstraction by the FRAGS reagent (benzyl BDE ˜90 kcal/mol). Although the BDE values calculated at the three theory levels are different, it is clear that the trends for relative energies are quite similar. A more thorough potential energy surface exploring subtleties in conformation and interaction would be necessary to compare the relative reactivities of each carbon site and estimate the activation energies, which are likely to be significantly in excess of the small 4-6 kcal/mol endothermicity. In addition, the relative abundance of product ions in FRAGS spectra induced by free radical chemistry is slightly higher than those resulting from acid-base chemistry, suggesting that the activation energies for free radical-driven processes are slightly lower than those for acid-base chemistry. However, reasonable yields of both types of product ions are generally observed.

### Example 7

### Materials

Glycans were purchased from Sigma-Aldrich (St. Louis, Mo., USA). All solvents are HPLC grade and were purchased from EMD Merck (Gibbstown, N.J., USA). All other chemicals were purchased from Sigma-Aldrich (St. Louis, Mo., USA). For desalting, OMIX 100 μL size C-18 tips were purchased from Varian Inc. (Palo Alto, Calif., USA).

### Example 8

### Synthesis of FRAGS and PRAGS Reagents

### Methyl 5-(bromomethyl)nicotinate (4)

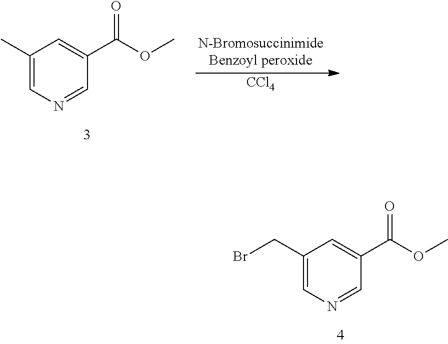

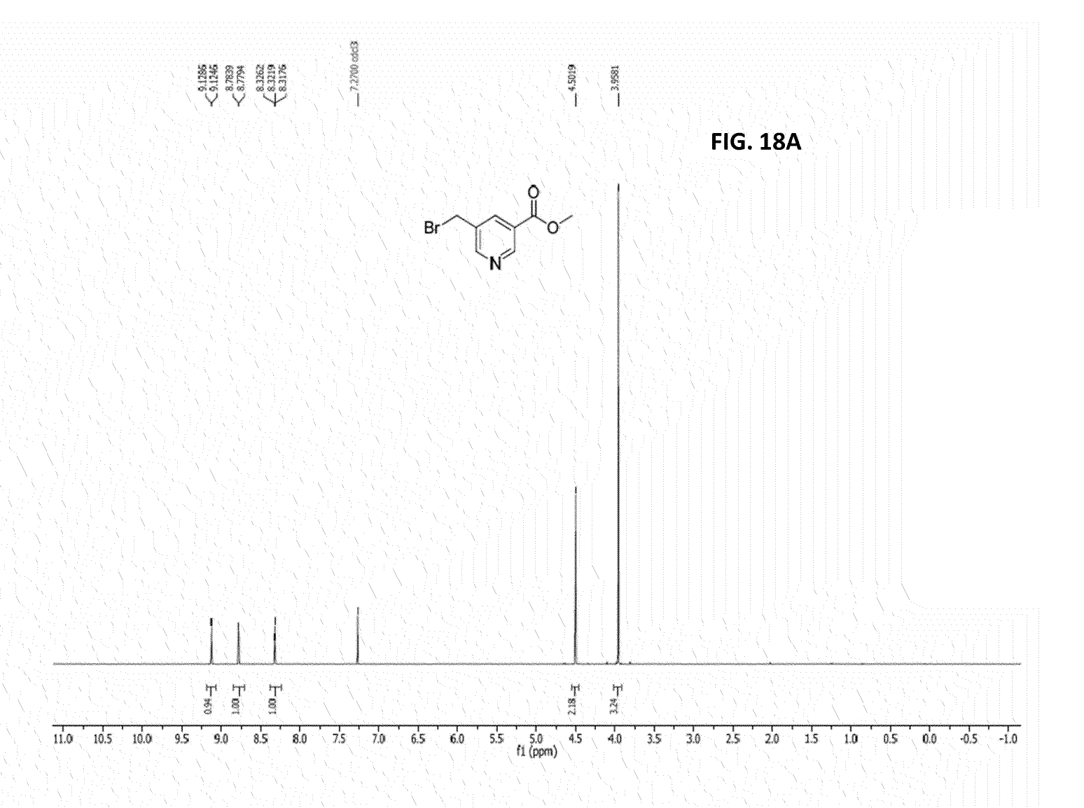

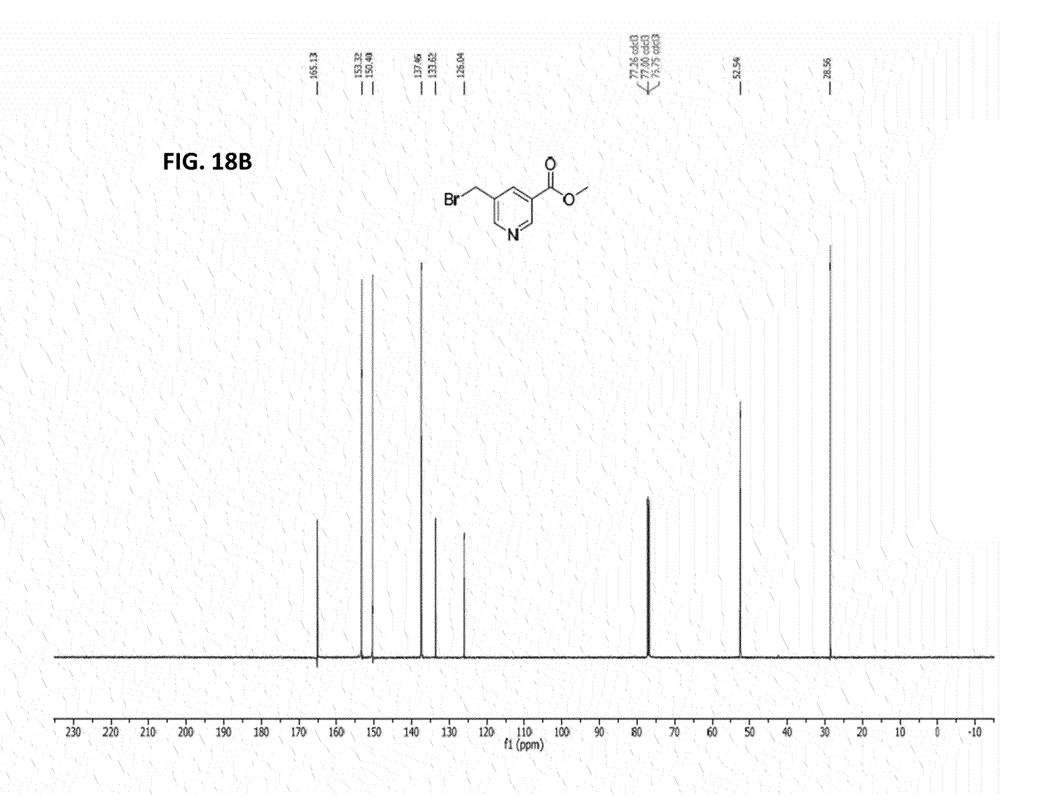

To a solution of methyl 5-methylnicotinate (3) (1.51 g, 10 mmol) and N-bromosuccinimide (NBS) (2.13 g, 12 mmol) in CCl₄ (50 mL) was added benzoyl peroxide (24.2 mg, 0.1 mmol) under argon. (Lee et al., Analyst 2009, 134, 1706-1712, the entire contents of which is herein incorporated by reference.) The reaction mixture was stirred under reflux for 10 hours. After cooling to room temperature, the reaction mixture was extracted with CH₂Cl₂ (×3). The extract was washed with brine, dried over anhydrous MgSO₄, filtered, and concentrated in vacuo. The crude product was purified by flash chromatography on silica gel (hexane-EtOAc 1:1) to give methyl 5-(bromomethyl)nicotinate (4) as a white solid (1.17 g, 51%). ¹HNMR (500 MHz, CDCl₃, δ ppm): 9.13 (d, J=2.0 Hz, 1H), 8.78 (d, J=2.2 Hz, 1H), 8.32 (t, J=2.2 Hz, 1H), 4.50 (s, 2H), 3.96 (s, 3H).); ¹³C NMR (125 MHz, CDCl₃, δ ppm). 28.6, 52.5, 126.0, 133.6, 137.5, 150.4, 153.3, 165.1. ESI-MS: [M+H]⁺, 230.0, 232.0 (FIGS. 18A, 18B).

### Methyl 5-(((2,2,6,6-tetramethylpiperidin-1-yl)oxy)methyl)nicotinate (5)

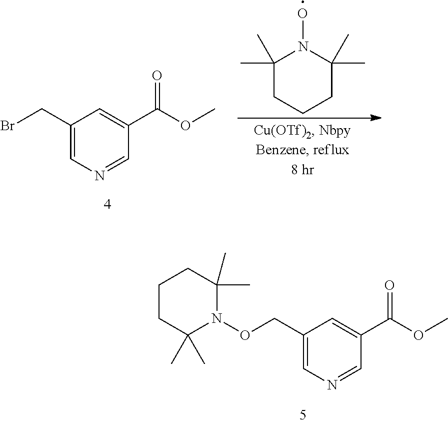

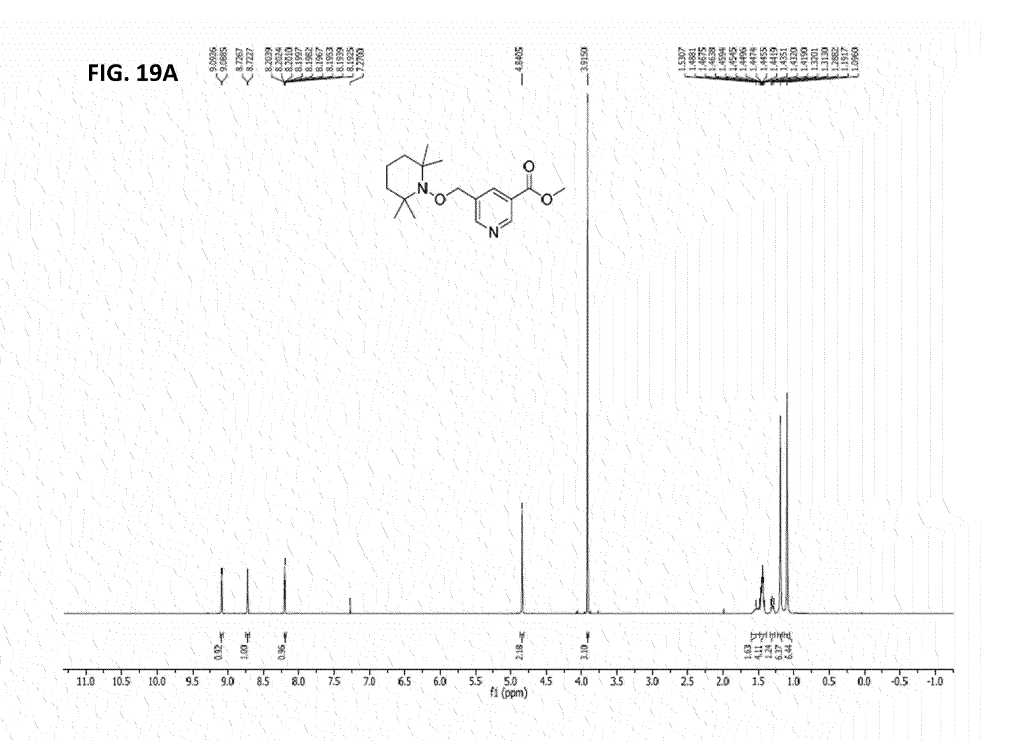

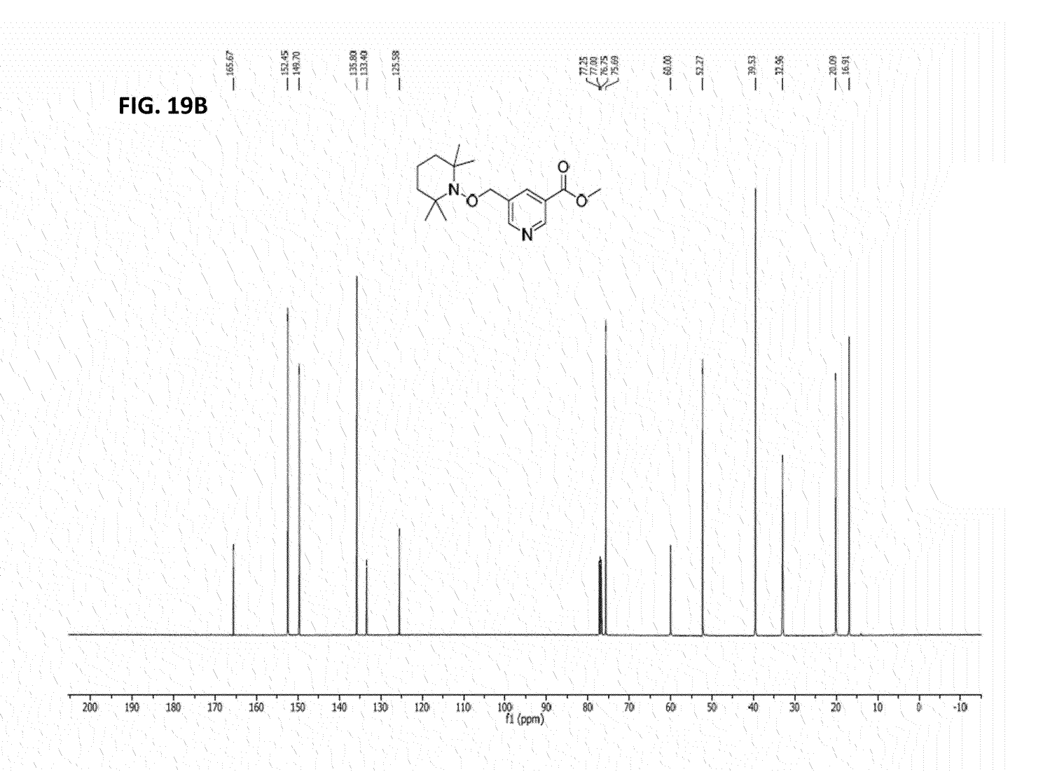

To a Schlenk flask was added 4 (1.15 g, 5 mmol), (2,2,6,6-tetramethylpiperidin-1-yl)oxyl (TEMPO, 936 mg, 6 mmol), Cu(OTf)₂ (217 mg, 0.6 mmol), copper powder (32.0 mg, 5 mmol), 4,4′-dinonyl-2,2′-bipyridyl (Nbpy, 818 mg, 2 mmol), and benzene (15 mL). (Lee et al., supra.) The reaction mixture was degassed by bubbling argon for 5 min and heated at 80° C. overnight. After cooling the reaction mixture to room temperature, it was filtered through a short pad of silica gel using EtOAc. The filtrate was concentrated in vacuo and the residue was purified by flash column chromatography using hexane-EtOAc (3:1). The desired product 5 was obtained as a white solid (1.16 g, 76%). ¹HNMR (500 MHz, CDCl₃, δ ppm): 9.09 (d, J=2.1 Hz, 1H), 8.70 (d, J=2.0 Hz, 1H), 8.20 (m, 1H), 4.84 (s, 2H), 3.92 (s, 3H), 1.53 (m, 1H), 1.45 (m, 4H), 1.30 (m, 1H), 1.19 (s, 6H), 1.10 (s, 6H); ¹³C NMR (125 MHz, CDCl₃, δ ppm), 16.9, 20.1, 32.7, 39.5, 52.3, 60.0, 75.7, 125.6, 133.4, 135.8, 149.7, 152.5, 165.7. ESI-MS: [M+H]⁺, 307.3 (FIGS. 19A, 19B).

### (5-(((2,2,6,6-Tetramethylpiperidin-1-yl)oxy)methyl)pyridin-3-yl)methanol (6)

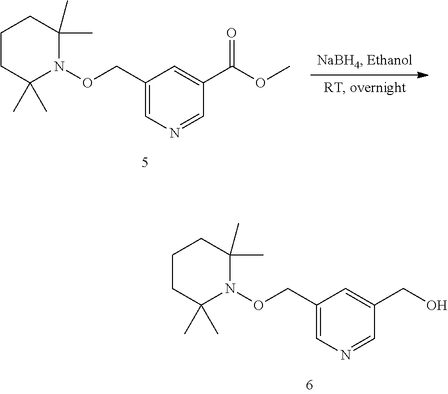 [SMILES: COC(=O)c1cncc(CON2C(C)(C)CCCC2(C)C)c1 ; CC1(C)CCCC(C)(C)N1OCc1cncc(CO)c1]

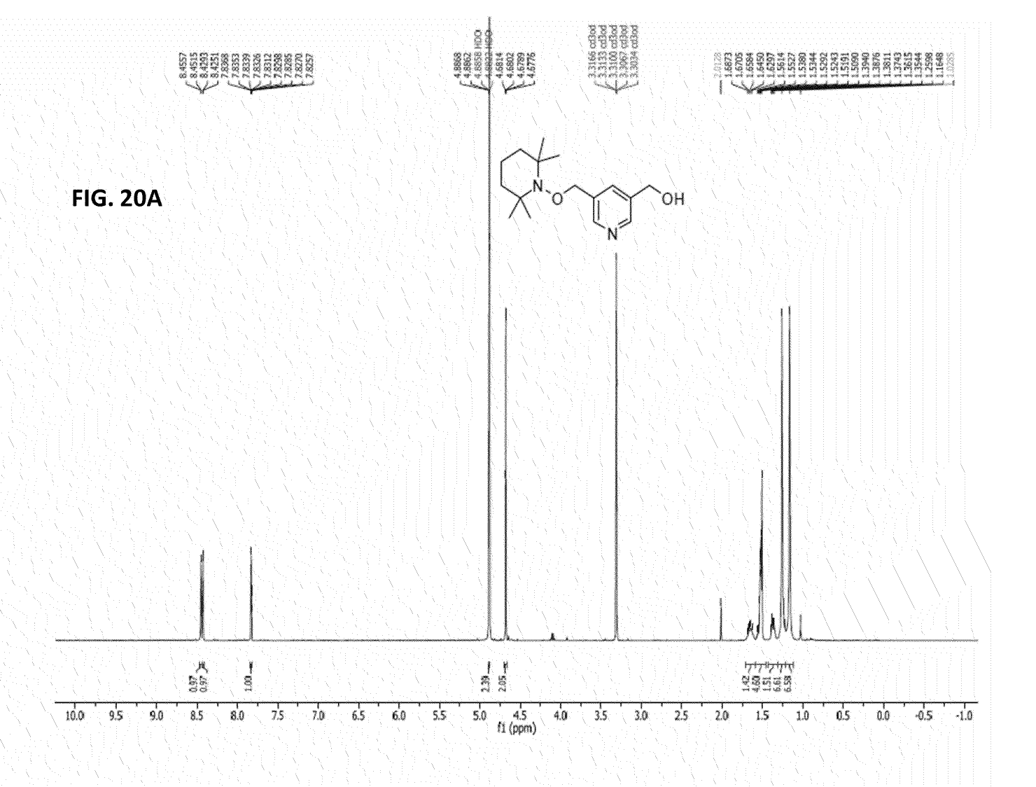

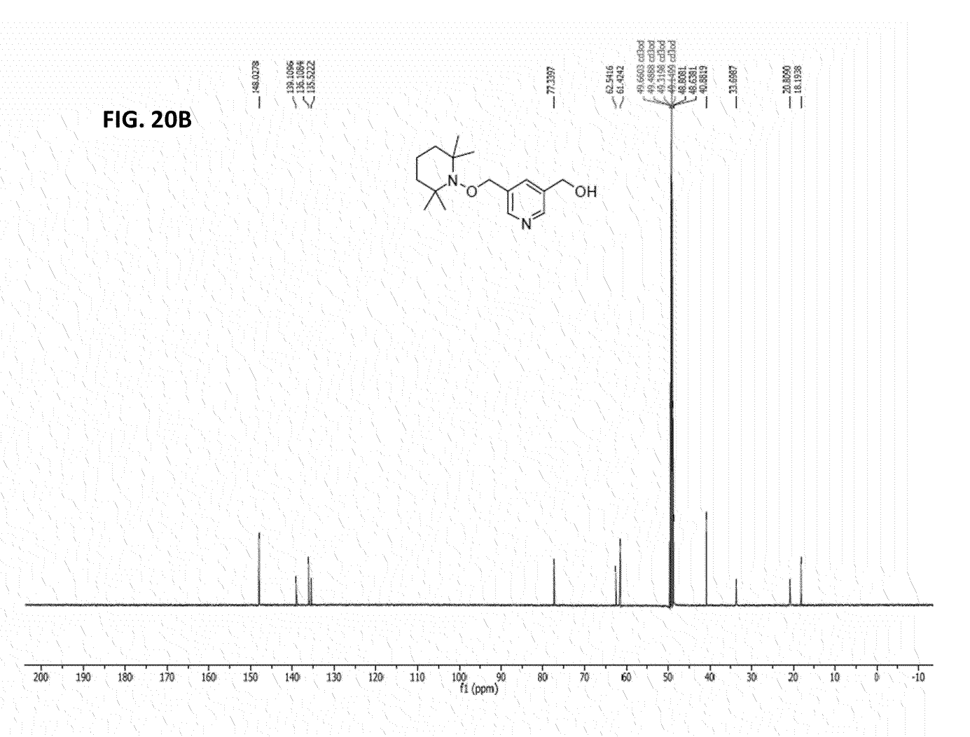

To a solution of 5 (1.07 g, 3.5 mmol) in anhydrous ethanol (20 mL) was added sodium borohydride (266 mg, 7 mmol) portionally under argon. (WO/2008/005457, the entire contents of which are herein incorporated by reference.) The reaction mixture was stirred under reflux overnight. After cooling to room temperature, the reaction mixture was diluted by adding 50 ml EtOAc, washed by water and brine, dried over Na₂SO₄, filtered, and concentrated in vacuo. The crude product was purified by flash chromatography on silica gel (hexane-EtOAc 1:1 to 1:5) to give the desired product 6 as a white solid (731 mg, 75%). ¹HNMR (500 MHz, CD₃OD, δ ppm): 8.45 (d, J=2.1 Hz, 1H), 8.43 (d, J=2.1 Hz, 1H), 7.83 (m, 1H), 4.89 (s, 2H), 4.68 (m, 2H), 1.66 (m, 1H), 1.54 (m, 4H), 1.38 (m, 1H), 1.26 (s, 6H), 1.16 (s, 6H); ³C NMR (125 MHz, CD₃OD, δ ppm), 18.2, 20.8, 33.7, 40.9, 61.4, 62.5, 77.3, 135.5, 136.1, 139.1, 148.0. ESI-MS: [M+H]⁺, 279.3 (FIGS. 20A, 20B).

### 2-((5-(((2,2,6,6-Tetramethylpiperidin-1-yl)oxy)methyl)pyridin-3-yl)methoxy)isoindoline-1,3-dione (8)

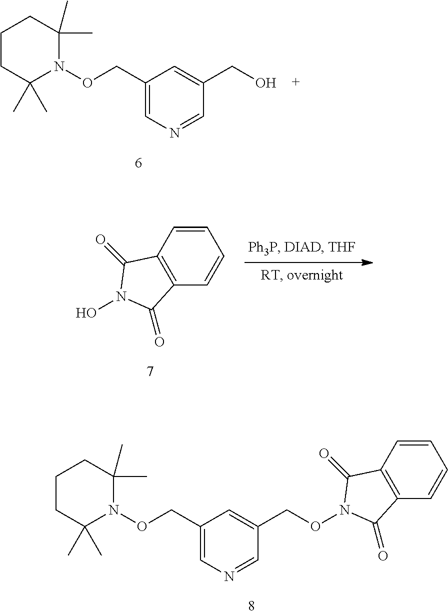 [SMILES: CC1(C)CCCC(C)(C)N1OCc1cncc(CO)c1 ; O=C1c2ccccc2C(=O)N1O ; CC1(C)CCCC(C)(C)N1OCc1cncc(CON2C(=O)c3ccccc3C2=O)c1]

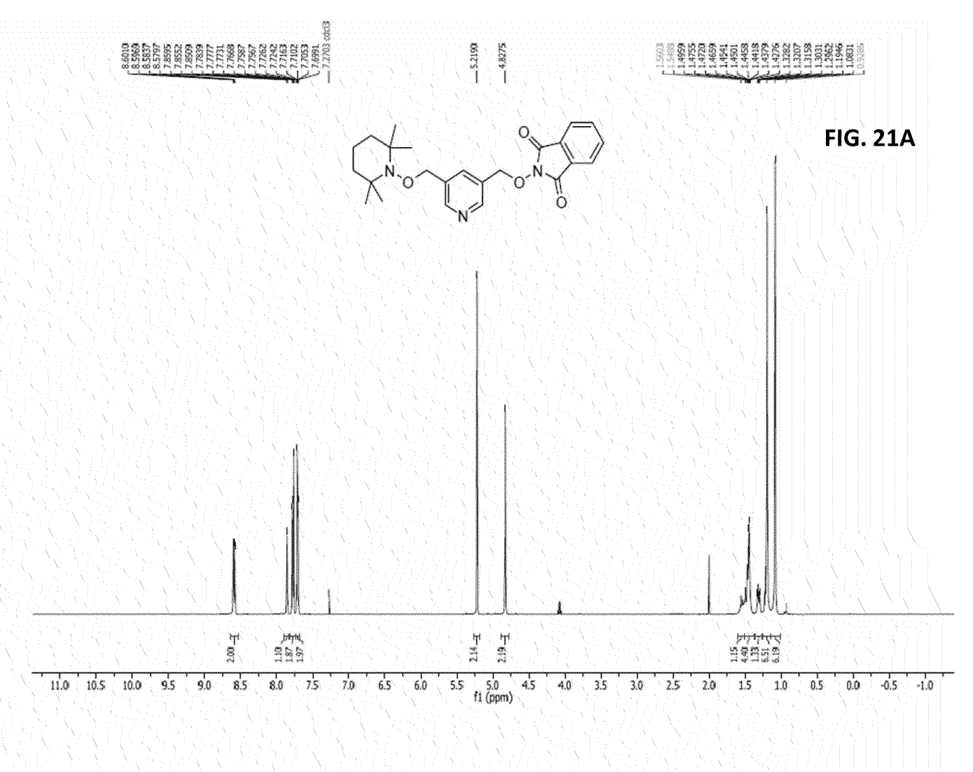

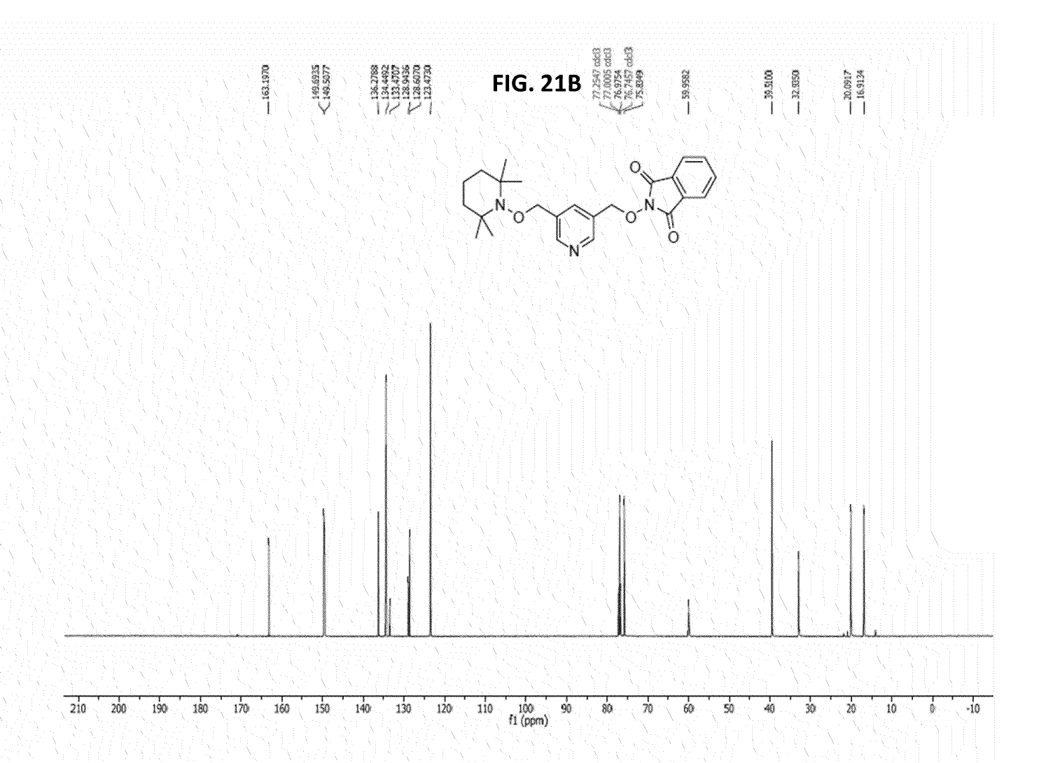

To a solution of 6 (557 mg, 2 mmol), 2-hydroxyisoindoline-1,3-dione (7, 391 mg, 2.4 mmol), and triphenylphosphine (629 mg, 2.4 mmol) in anhydrous THF (20 mL) was added a solution of diisopropyl azodicarboxylate (DIAD, 525 mg, 2.6 mmol) in 5 ml anhydrous THF at 0° C. under argon. (Deraeve et al., J. Am. Chem. Soc. 2012, 134, 7384-7391, the entire contents of which are herein incorporated by reference.) The reaction mixture was stirred at 0° C. for 30 minutes and then warmed to room temperature and stirred overnight. The reaction mixture was washed by water and brine, dried over Na₂SO₄, filtered, and concentrated in vacuo. The crude product was purified by flash chromatography on silica gel (hexane-EtOAc 1:2) to give the desired product 8 as a white solid (695 mg, 82%). ¹HNMR (500 MHz, CDCl₃, δ ppm): 8.60 (d, J=2.1 Hz, 1H), 8.58 (d. J=2.0 Hz, 1H), 7.86 (t, J=2.1 Hz, 1H), 7.78 (dd, J₁=2.1 Hz, J₂=5.5 Hz, 2H), 7.71 (dd, J₁=2.1 Hz, J₂=5.5 Hz, 2H), 5.22 (s, 2H), 4.83 (s, 2H), 1.55 (m, 1H), 1.45 (m, 4H), 1.32 (m, 1H), 1.26 (s, 6H), 1.15 (s, 6H); ¹³C NMR (125 MHz, CDCl₃, δ ppm), 16.9, 20.1, 32.9, 39.5, 60.0, 75.8, 77.0, 123.5, 128.6, 128.9, 133.5, 134.5, 136.3, 149.5, 149.7, 163.2. ESI-MS: [M+H]⁺, 424.4 (FIGS. 21A, 21B).

### O-((5-(((2,2,6,6-tetramethylpiperidin-1-yl)oxy)methyl)pyridin-3-yl)methyl)hydroxylamine (FRAGS II, 2)

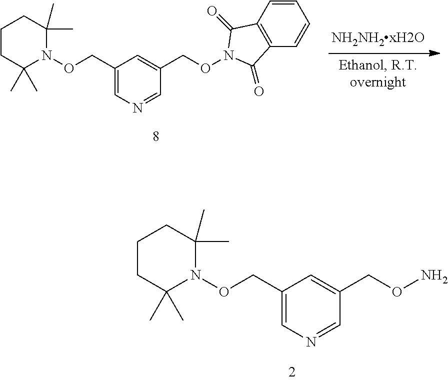 [SMILES: CC1(C)CCCC(C)(C)N1OCc1cncc(CON2C(=O)c3ccccc3C2=O)c1 ; CC1(C)CCCC(C)(C)N1OCc1cncc(CON)c1]

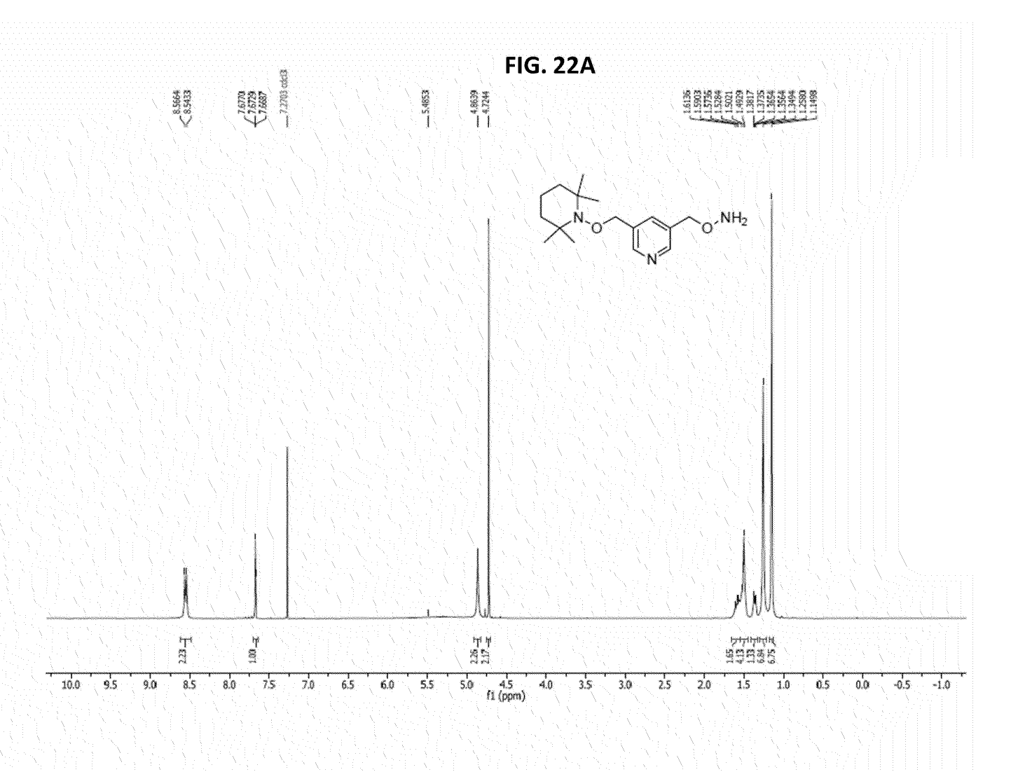

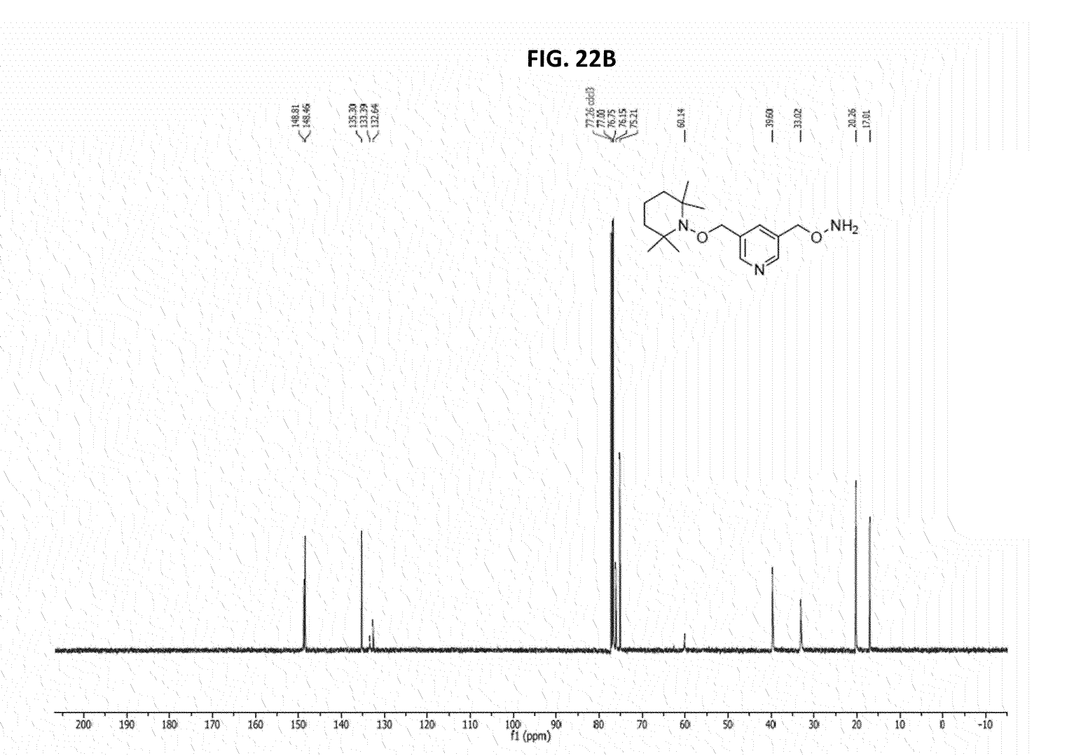

To a solution of 8 (1.07 g, 1.5 mmol) in anhydrous ethanol (20 mL) was added hydrazine hydrate (N₂H₄, 50-60%, 0.86 mL, 15 mmol) portionally under argon. (Deraeve et al., supra.) The reaction mixture was stirred under reflux overnight. After cooling to room temperature, the reaction mixture was concentrated in vacuo. The crude product was purified by flash chromatography on silica gel (CHCl₃-Hexane 3:1) to give the desired product 2 as a white solid (378 mg, 86%). ¹HNMR (500 MHz, CDCl₃, δ ppm): 8.57 (s, 1H), 8.54 (s, 1H), 7.67 (t, J=2.1 Hz, 1H), 4.86 (s, 2H), 4.72 (s, 2H), 1.59 (m, 1H), 1.50 (m, 4H), 1.37 (m, 1H), 1.26 (s, 6H), 1.15 (s, 6H); ¹³C NMR (125 MHz, CDCl₃, δ ppm), 17.0, 20.3, 33.0, 39.6, 60.1, 75.2, 76.2, 132.6, 133.4, 135.3, 148.5, 148.8. ESI-MS: [M+H]⁺, 294.4 (FIGS. 22A, 22B).

### O-(pyridin-3-ylmethyl)hydroxylamine (PRAGS, 1)

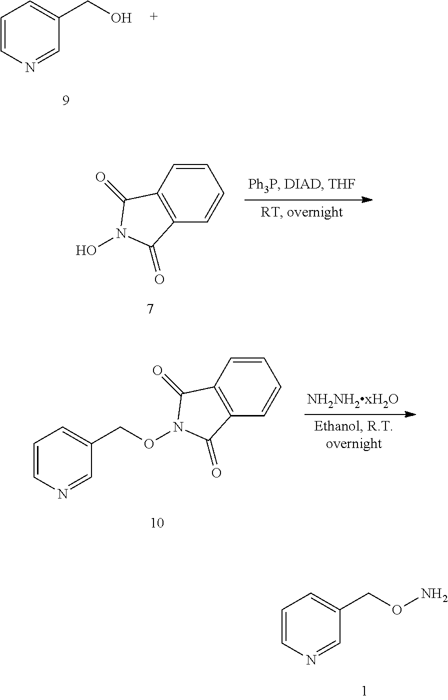 [SMILES: OCc1cccnc1 ; O=C1c2ccccc2C(=O)N1O ; O=C1c2ccccc2C(=O)N1OCc1cccnc1 ; NOCc1cccnc1]

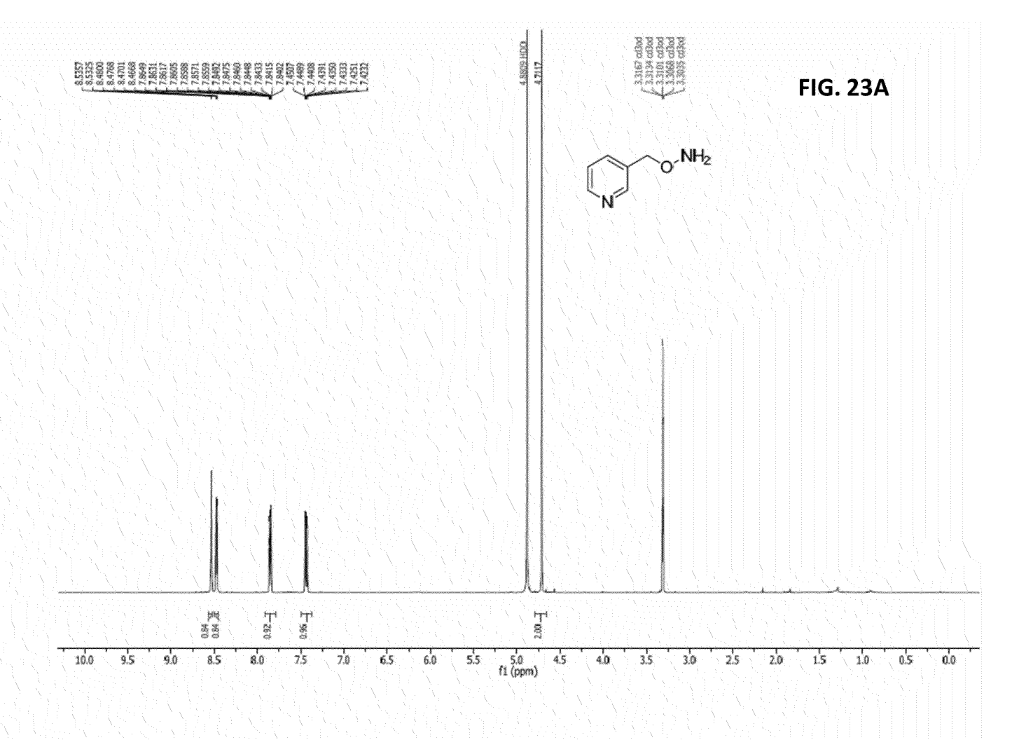

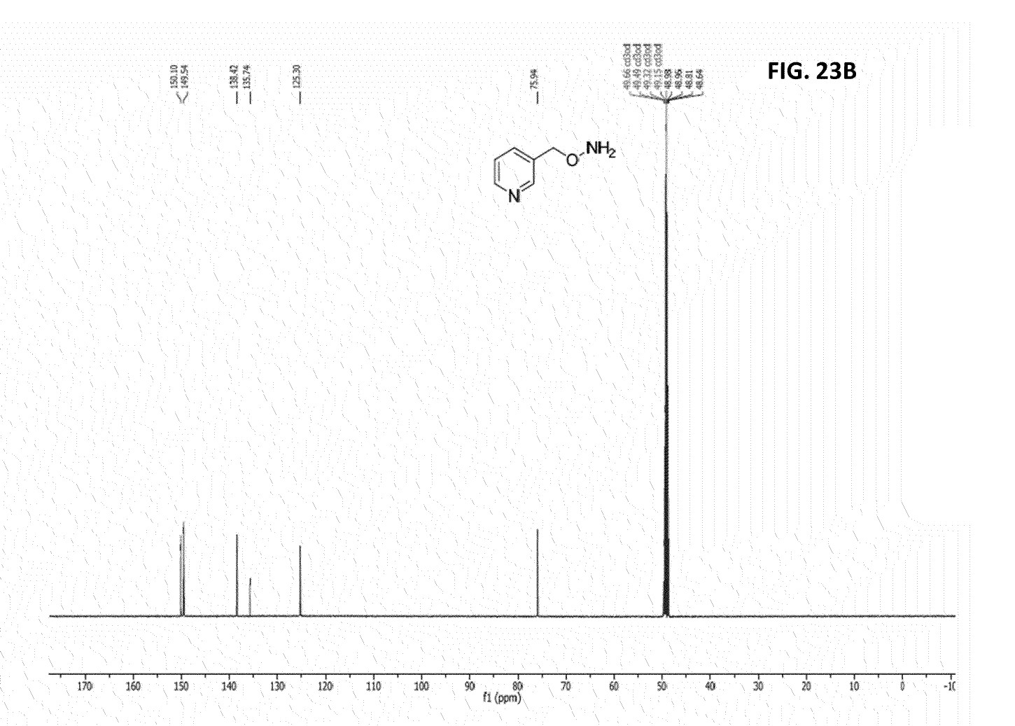

O-(pyridin-3-ylmethyl)hydroxylamine (PRAGS, 1) was synthesized following the procedure for synthesis of FRAGS reagent (2). (Deraeve et al., supra.) Overall yield 46%. ¹HNMR (500 MHz, CD₃OD, δ ppm): 8.54 (d, 1H), 8.47 (dd, J₁=1.6 Hz, J₂=5.0 Hz, 1H), 7.85 (m, 1H), 7.44 (m, 1H), 4.71 (s, 2H); ¹³C NMR (125 MHz, CDCl₃, δ ppm), 75.4, 125.3, 135.7, 138.4, 149.5, 150.1. ESI-MS: [M+H]⁺, 125.1 (FIGS. 23A, 23B).

### Example 9

### Procedure Steps for Purification of PRAGS and FRAGS Derivatized Glycans Using C18 Pipet Tip

1. Attach a C18 pipet tips to a micropipettor and condition twice by aspirating 10 μl of 1:1 acetonitrile/water. 2. Wash pipet tip twice with 10.1 water. 3. Draw up 10 μl of aqueous glycan derivatization solution and return to the main solution. Repeat approximately ten times to saturate the C18 pipet tip with the derivatized glycan. 4. Wash pipet tip twice with 10 μl water. 5. Draw up 10 μl of 1:1 acetonitrile/water and return to the main solution. Repeat approximately ten times to elute glycans. The solution was used directly for ESI-MS.

### Example 10

### Mass Spectrometry

A Thermo-Fisher Scientific linear quadrupole ion trap (LTQ-XL) mass spectrometer (Thermo, San Jose, Calif., USA) equipped with an electrospray ionization (ESI) source was employed in experiments with PRAGS, FRAGS I and FRAGS II. Derivatized glycan sample solutions were directly infused to the ESI source of the mass spectrometer via a syringe pump at a flow rate of 3 μL/min. Critical parameters of the mass spectrometer include spray voltage of 5˜6 kV, capillary voltage of 30˜40 V, capillary temperature of 275° C., sheath gas (N2) flow rate of 8˜10 (arbitrary unit), and tube lens voltage of 50˜200 V. Other ion optic parameters were optimized by the auto-tune function in the LTQ-XL tune program for maximizing the signal intensity. CID was performed by resonance excitation of the selected ions for 30 ms. The normalized CID energy was 7˜30 (arbitrary unit).

### Example 11

### Quantum Chemical Calculation

The molecule α-1-O-methyl-D-glucopyranose was used as a simple model system to calculate key bond dissociation enthalpies (BDE) of glycans used in this study, where BDE in this work refers to the bond dissociation enthalpy at 298 K. Initial geometries of the monosaccharide were generated by the MC/MM conformer search with the OPLS 2005 force field using Macromol 8.0 (Schrödinger Inc., Portland, Oreg., USA) implemented in Maestro 8.0 (Schrödinger Inc., Portland, Oreg., USA) under the Linux environment. Within 5 kcal/mol energy, all low energy conformers were initially recorded. After manual screening of obtained structures to avoid redundancy, low energy conformers were selected for further structure optimization by density functional theory (DFT). Each conformer was subject to a geometry optimization using Jaguar 7.5 (Schrödinger Inc., Portland, Oreg. USA) at the B3LYP/6-31+G(d) level. The lowest-energy structure was then utilized as a starting point for optimization of the radical species of interest utilizing DFT at the same level of theory. The single point energy for each species was then refined within Jaguar using B3LYP, M05-2X, and M06-2X density functionals at the 6-311++G(d,p) level of theory with the spin-unrestricted method. The two new generation meta-hybrid functionals other than B3LYP were chosen for their ability to more reliably predict the energetics of organic radical reactions. Thermochemical corrections (zero-point energy and enthalpy) were obtained utilizing the B3LYP/6-311++G(d,p) level of theory and applied to all density functionals for calculation of the bond dissociation enthalpy at 298 K. All calculations were performed using computational resources kindly provided by the Material and Process Simulation Center at the Beckman Institute, Caltech.

BDEs were determined via the isodesmic method, in which the BDE of a reference molecule is utilized to determine the unknown BDE. To determine the BDE of C—H bonds in the monosaccharide, the enthalpy of reaction for hydrogen atom transfer between a carbon-centered methanol radical and each carbon in the sugar was calculated. The use of a reference molecule similar to the compound under study reduces any systematic error from differences between the two species. For the determination of O—H BDEs, an oxygen-centered isopropanol radical was utilized in place of the methanol radical.

As disclosed throughout and evidenced by the data presented in the accompanying figures, for example, FIGS. 4A-4F, FIGS. 6A-6L, FIGS. 11A-11F, and FIGS. 16A-16C, the compositions of the present invention provide a means for determining the glycan sequence of a glycan having a reducing terminus.

While the present invention has been illustrated and described with reference to certain exemplary embodiments, those of ordinary skill in the art will understand that various modifications and changes may be made to the described embodiments without departing from the spirit and scope of the present invention, as defined in the following claims.

What is claimed is:

1. A glycan reagent for depolymerization of a glycan having a reducing end, the glycan reagent being represented by the formula MCX, wherein M is a glycan coupling group selected from oxylamines and hydrazides; C is a fixed charge group or a basic group having a proton affinity of at least 210 kcal/mol; and X is hydrogen or a free radical initiator chemically coupled to C.

2. The glycan reagent of claim 1, wherein C is selected from the group consisting of cationic species with and without a labile proton, anionic species that have conjugate bases of neutral acids, and anionic species having a fixed negative charge.

3. The glycan reagent of claim 1, wherein the free radical initiator is selected from the group consisting of peroxides, nitrites, alkoxyamines, and azo compounds.

4. The glycan reagent of claim 1, wherein the free radical initiator is selected from tetramethylpiperidin-1-yl)oxy (TEMPO) or 4,4-azobis(4-cyanopentanoic acid).

5. The glycan reagent of claim 1, wherein the glycan reagent is coupled to a resin support or an affinity tag.

6. The glycan reagent of claim 1, wherein the glycan reagent is represented by one of structures (a) through (k): 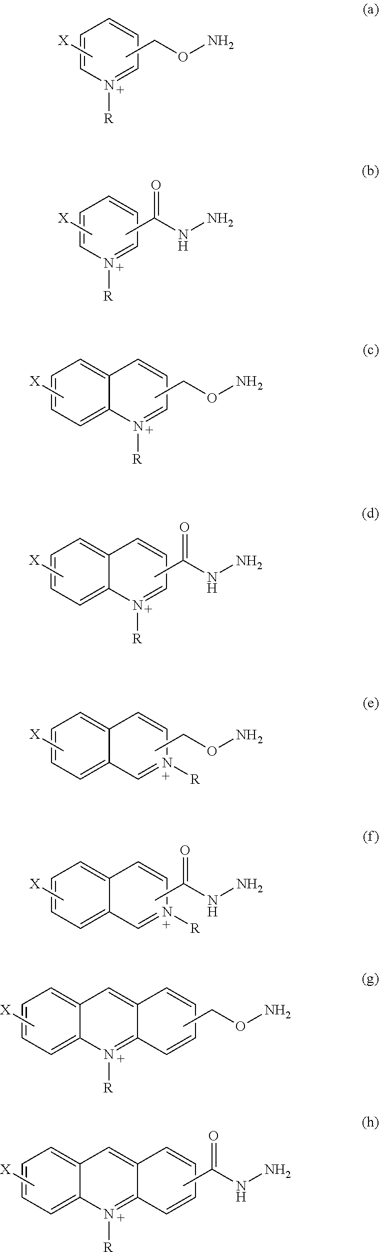 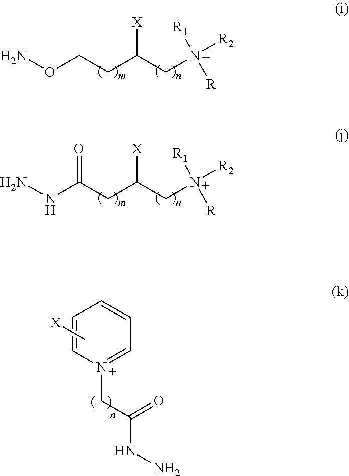
wherein X is hydrogen or a free radical initiator, R is hydrogen, an alkyl, or an alkoxy, R₁ and R₂ are each independently an alkyl, and m and n are each independently an integer.

7. The glycan reagent of claim 6, wherein when X is hydrogen, R is hydrogen.

8. The glycan reagent of claim 6, wherein the free radical initiator is TEMPO-CH₂.

9. A method of depolymerizing a glycan having a reducing terminus, comprising:
conjugating the glycan reagent of claim 1 to the reducing terminus of the glycan to form a derivatized glycan;
ionizing the derivatized glycan to form an ionized derivatized glycan; and
dissociating the ionized derivatized glycan to form fragment glycan ions.

10. The method of claim 9, wherein the ionizing comprises electrospray ionization.

11. The method of claim 9, wherein the dissociating comprises collision-induced dissociation.

12. The method of claim 9, further comprising analyzing the fragment glycan ions.

13. The method of claim 12, wherein the analyzing is performed in a mass spectrometer.

14. A method of depolymerizing a glycan having a reducing terminus, comprising:
conjugating the glycan reagent of claim 5 to the reducing terminus of the glycan to form a derivatized glycan;
isolating the derivatized glycan to form an isolated derivatized glycan;
ionizing the isolated derivatized glycan to form an ionized derivatized glycan; and
dissociating the ionized derivatized glycan to form fragment glycan ions.

15. A method of identifying subunit connectivity of a glycan having a reducing terminus, comprising:
conjugating a glycan reagent represented by one of structures (a) through (j) to the reducing terminus of the glycan to form a derivatized glycan 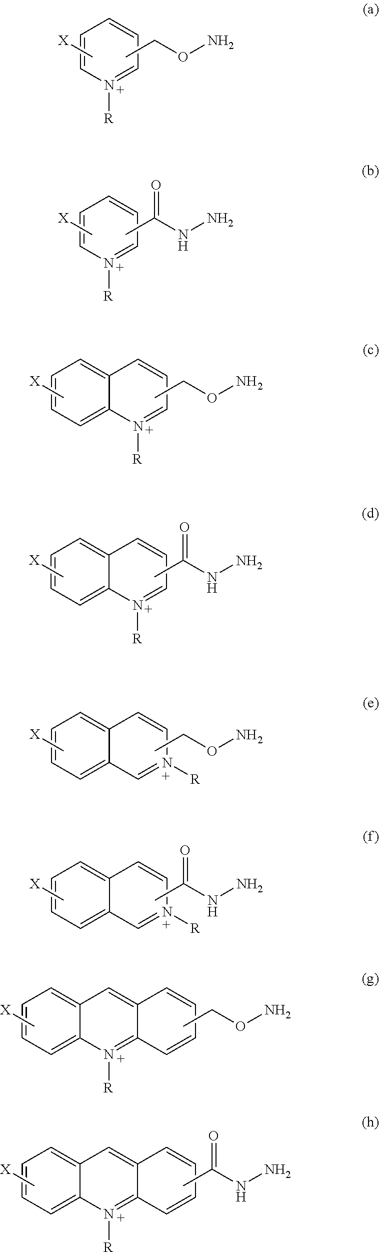 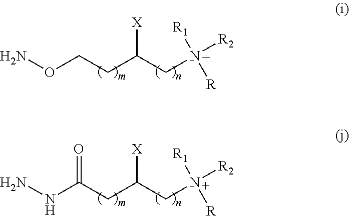
wherein X is hydrogen, R is hydrogen, R₁ and R₂ are each independently an alkyl;
ionizing the derivatized glycan to form an ionized derivatized glycan;
dissociating the ionized derivatized glycan to form fragment glycan ions; and
analyzing the fragment glycan ions to determine the subunit connectivity of the glycan.

16. The method of claim 15, further comprising dissociating the fragment glycan ions to form other fragment glycan ions.

17. The method of claim 16, wherein the analyzing the fragment glycan ions further comprises analyzing the other fragment glycan ions.
